SEQUENCE LISTING (13 sequences)

SEQ ID NO: 1 - protein, 5 aa, Artificial
  avelumab H-CDR1
SYIMM

SEQ ID NO: 2 - protein, 17 aa, Artificial
  avelumab H-CDR2
SIYPSGGITFYADTVKG

SEQ ID NO: 3 - protein, 11 aa, Artificial
  avelumab H-CDR3
IKLGTVTTVDY

SEQ ID NO: 4 - protein, 14 aa, Artificial
  avelumab L-CDR1
TGTSSDVGGYNYVS

SEQ ID NO: 5 - protein, 7 aa, Artificial
  avelumab L-CDR2
DVSNRPS

SEQ ID NO: 6 - protein, 10 aa, Artificial
  avelumab L-CDR3
SSYTSSSTRV

SEQ ID NO: 7 - protein, 449 aa, Artificial
  avelumab heavy chain (without C-terminal lysine)
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 450 aa, Artificial
  avelumab heavy chain variant with C-terminal lysine
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 216 aa, Artificial
  avelumab light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 10 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 11 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 12 - protein, 243 aa, Artificial
  HPV-16 SS-E6*-TMF
MGLKVNVSAIFMAVLLTLQTPTGQIHWGMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLQRHLDKKQRFHNIRGRWTGRCMSCCRSSRTRRETQLGLSSTSIVYILIAVCLGGLIGIPALICCCRGRCNKKGEQVGMSRPGLKPDLTGTSKSYVRSL

SEQ ID NO: 13 - protein, 185 aa, Artificial
  HPV-16 SS-16E7*-TMR
MVPQALLFVPLLVFPLCFGKFPIGSMHGDTPTLHEYMLDLQPETTQLNDSSEEEDEIDGPAGQAEPDRAHYNIVTFCCKCDSTLRLCVQSTHVDIRTLEDLLMGTLGIVCPICSQKPRSYVLLSAGALTALMLIIFLMTCCRRVNRSEPTQHNLRGTGREVSVTPQSGKIISSWESHKSGGETRL

(19) United States
(12) Patent Application Publication
(10) Pub. No.: US 2023/0405105 A1
(43) Pub. Date: Dec. 21, 2023

(54) COMBINATION OF A POXVIRUS ENCODING HPV POLYPEPTIDES WITH AN ANTI-PD-L1 ANTIBODY

(71) Applicants: TRANSGENE SA, Illkirch-Graffenstaden Cedex (FR); MERCK PATENT GmbH, Darmstadt (DE); PFIZER INC., New York, NY (US)

(72) Inventors: Kaidre BENDJAMA, Strasbourg (FR); Maud BRANDELY-TALBOT, Strasbourg (FR); Annette TAVERNARO, Ottrott (FR)

(21) Appl. No.: 17/753,943

(22) PCT Filed: Sep. 21, 2020
(86) PCT No.: PCT/EP2020/076232

§ 371 Date: Mar. 18, 2022

(30) Foreign Application Priority Data
Sep. 20, 2019 (EP) 19306159.5
Jun. 24, 2020 (EP) 20305697.3

(51) Int. Cl. A61K 39/12; A61K 39/395; A61P 35/04

(52) U.S. Cl. CPC A61K 39/12; A61K 39/3955; A61P 35/04; A61K 2039/5256

(57) ABSTRACT

The present invention relates to a combination of a) a poxvirus vector encoding at least human papillomavirus (HPV) E6 and E7 polypeptides and an immunostimulatory cytokine, and b) an anti-PD-L1 antibody or antigen-binding fragment thereof, for use in the treatment of an HPV-positive cancer, wherein a first administration of said poxvirus is performed 5 to 10 days before the first administration of said anti-PD-L1 antibody, and subsequent administrations of said poxvirus and anti-PD-L1 antibody are performed.

### TECHNICAL FIELD OF THE INVENTION

The present invention relates to a combination of a) a poxvirus vector encoding at least human papillomavirus (HPV) E6 and E7 polypeptides and an immunostimulatory cytokine, and b) an anti-PD-L1 antibody or antigen-binding fragment thereof, for use in the treatment of an HPV-positive cancer, wherein a first administration of said poxvirus is performed 5 to 10 days before the first administration of said anti-PD-L1 antibody, and subsequent administrations of said poxvirus and anti-PD-L1 antibody are performed.

### BACKGROUND ART

Human papillomavirus (HPV) is the most common diagnosed sexually transmitted infection. It is associated with condyloma acuminata, anogenital (cervical, vaginal, vulval, penile, anal) squamous intraepithelial lesions and malignancies, and also squamous cell carcinoma of the head and neck (SCCHN).

HPV is a small deoxyribonucleic acid (DNA) virus of approximately 7900 base pairs. The HPV genome encodes DNA sequences for six early (E) proteins associated with viral gene regulation and cell transformation, two late proteins which form the shell of the virus, and a region of regulatory DNA sequences known as the long control region or upstream regulatory region (Palefsky J. M. and Holly E. A., Cancer Epidemiol Biomarkers Prev. (1995) 4(4): 415-428).

HPV genotypes can be broadly split into “high-risk” (16, 18, 31, 33, 35, 39, 45, 51, 52, 56, 58, 59 and 68) and “low-risk” (6, 11, 40, 42, 43, 44, 53, 54, 61, 72, 73 and 81) based upon their malignant potential. Types 16 and 18 are the most commonly found HPV types in cancer with type 16 found in approximately 50% of patients with cervical cancer for example. Beyond causing cervical cancer, HPV is also implicated in cancer of the anus and penis. There is also an approximately 2 to 3 fold increased risk for cancers of the oral cavity and oropharynx in patients infected with high-risk (oncogenic) HPV subtypes: HPV-16 genotype but also HPV-18, 31 or 33. HPV associated tumors predominantly arise in the base of the tongue or the tonsillar region, although a small percentage of tumors at other sites are also HPV-positive. It is unclear why the oropharynx is more susceptible to HPV transformation than other sites.

A review on the published clinical trials, both single-arm and randomized, demonstrated HPV positivity ranging from 20 to 60% of oropharyngeal cancers (Ihloff A. S. et al., Oral Oncol. (2010) 46(10): 705-711).

Not only is high-risk HPV infection associated to various cancers, but it is also known to be involved in carcinogenesis of HPV-positive cancers. In particular, studies show that an infection with high-risk HPV is an independent risk factor for the development of SCCHN and needs to be considered along with the traditional risk factors such as tobacco abuse or alcohol (D'Souza G. et al., The New England journal of Medicine. (2007) 356(19): 1944-1956).

The mechanisms linking HPV infection with carcinogenesis has been shown to be linked to two viral proteins E6 and E7. E6 and E7 are described as oncoproteins as they have the capacity to disrupt normal replication control of the infected cells by inhibiting key regulation factors. The E6 oncoprotein binds and induces the degradation of the p53 tumor suppressor protein via an ubiquitin-mediated process disrupting the p53 pathway which leads to uncontrolled cell cycle progression (Chung C. H. and Gillison M. L., Clin Cancer Res. (2009) 15(22): 6758-6762). The HPV E7 protein binds and inhibits the retinoblastoma protein (pRb), preventing it from inhibiting the transcription factor E2F resulting in loss of cell cycle control. Furthermore, the functional inactivation of Rb results in upregulation of the p16-protein. P16 is encoded by the CDKN2A tumor suppressor gene and regulates the activity of Cyclin D-CDK4/6 complexes that phosphorylate Rb leading to release of the transcription factor E2F which initiates cell cycle progression.

Two prophylactic vaccines have been developed against HPV infection; one is a quadrivalent vaccine (Gardasil) and the other is a bivalent vaccine (Cervarix). Both are approved for the prevention of HPV infection and HPV related diseases in subjects that did not experience prior exposition to HPV.

To date, however, few antitumor vaccines for HPV-positive cancers, in particular SCCHN, have been clinically evaluated. Vaccine-driven generation and long-term persistence of tumor-specific immune cells are the objectives that antitumor vaccines have to realize to be clinically beneficial in HPV-positive cancers, in particular SCCHN.

TG4001 (corresponding to the research name MVATG8042) is a therapeutic recombinant vaccine/immunotherapy product based on the non-propagative highly attenuated vaccinia vector Modified Vaccinia virus Ankara (MVA) whose genome, a single linear double-stranded DNA molecule of approximately 178 kilobase pairs contains inserted transgenes coding for three proteins: HPV E6 and E7 onco-proteins modified to remove their oncogenic potential and human interleukin-2 (IL-2) as an adjuvant.

TG4001 was clinically investigated in gynaecological conditions. Among the 5 phase II studies carried out, four involved patients with precancerous lesions, specifically cervical intraepithelial neoplasia (CIN) grade 2/3 and vulvar intraepithelial neoplasia (VIN) grade 3, and one study included patients having cervical cancer. Two Phase II trials involving 21 (TG4001.07) and 206 (NV25025) patients with HPV-16 associated CIN grade 2/3 demonstrated a proof of concept that TG4001 had higher activity and efficacy compared to placebo in terms of histologic resolution and response rates as well as viral clearance. The dose that was used in these phase II trials of HPV-16 associated CIN grade 2/3 patients was 5×10⁷ Plaque Forming Unit (PFU) by subcutaneous route. With respect to the safety profile, the therapeutic vaccine product was demonstrated to be well tolerated (no major toxicities observed) with the most common adverse events being injection site reaction (Brun J. L. et al., Am J Obstet Gynecol. (2011) 204(2): 169 e161-168; Harper D. M. et al., Gynecol Oncol. (2019) 153(3): 521-529).

The overall safety profile of TG4001, based on data obtained from a total of 313 subjects (either healthy volunteers or patients with CIN 2/3, cervical carcinoma or VIN, treated by with TG4001 in monotherapy or in combination with immunomodulator imiquimod, via intramuscular or subcutaneous route) shows that TG4001 was well tolerated up to highest dose tested of 5×10⁷ PFU administered to patients either weekly or every 3 weeks up to 7 weeks, with a maximum of 6 injections. Concordant with its immunostimulatory nature, TG4001 administration is related to the onset of injection site reactions in most treated patients Most of these events were of mild to moderate intensity.

PD-1 is a negative regulator of T-cell activity that limits the activity of T cells at a variety of stages of the immune response when it interacts with its two ligands, PD-L1 and PD-L2. When engaged by a ligand, through phosphatase activity, PD-1 inhibits kinase-signaling pathways that normally lead to T-cell activation. A number of antibodies that disrupt the PD-1 axis have entered clinical development. PD-L1 is also believed to exert negative signals on T cells by interacting with B7, and PD-L1-blocking antibodies prevent this interaction. Immune checkpoint inhibitors also enhance the function of tumor-infiltrating lymphocytes (TILs), which augments antitumor immunity within the tumor microenvironment. The presence of TILs has been correlated with better prognosis in many cancer types. Thus, PD-L1⁺ TILs have been shown to be indicators of response to immune checkpoint blockade, and a lack of TILs may be a predictive marker for lack of response to PD-1/L1 blockade ((Herbst R. S. et al., Nature. (2014) 515(7528): 563-567).

Anti-PD-L1 antibodies have been clinically investigated in the treatment of various solid cancers and found to provide clinical benefit in various cancers (Brahmer J. R. et al., N Engl J Med. (2012) 366(26): 2455-2465).

Avelumab is a human anti-programmed death ligand-1 (PD-L1) antibody. Avelumab has been shown in preclinical models to engage both the adaptive and innate immune functions. By blocking the interaction of PD-L1 with PD-1 receptors, avelumab has been shown to release the suppression of the T cell-mediated antitumor immune response in preclinical models. Avelumab has also been shown to induce NK cell-mediated direct tumor cell lysis via antibody-dependent cell-mediated cytotoxicity (ADCC) in vitro. Avelumab in combination with axitinib is indicated in the US for the first-line treatment of patients with advanced renal cell carcinoma (RCC). The US Food and Drug Administration (FDA) also granted accelerated approval for avelumab for the treatment of (i) adults and pediatric patients 12 years and older with metastatic Merkel cell carcinoma (mMCC) and (ii) patients with locally advanced or metastatic urothelial carcinoma (mUC) who have disease progression during or following platinum-containing chemotherapy, or have disease progression within 12 months of neoadjuvant or adjuvant treatment with platinum-containing chemotherapy.

Avelumab has shown an acceptable safety profile in cancer patients. The warnings and precautions for avelumab include immune-mediated adverse reactions (such as pneumonitis and hepatitis [including fatal cases], colitis, endocrinopathies, nephritis and renal dysfunction and other adverse reactions [which can be severe and have included fatal cases]), infusion-related reactions, major adverse cardiovascular events (MACE), and embryo-fetal toxicity.

Clinical trials testing avelumab in HPV-positive cancers, such as SCCHN, are currently ongoing, but no results have been published at this stage.

Despite results obtained in precancerous lesions using TG4001 or in cancers using anti-PD-L1 antibodies alone, novel treatments of are still highly needed to improve anti-tumor efficacy and the proportion of responders. Accordingly, there remains a need to develop novel therapeutic options for the treatment of cancers. In particular, there exists a need for methods of treating cancer that improve the efficacy of (a) poxvirus vectors encoding at least human papillomavirus (HPV) E6 and E7 polypeptides and an immunostimulatory cytokine or (b) anti-PD-L1 antibodies in one or more types of cancer. The present invention provides a combination product for use in the treatment of HPV-positive cancer to address the above need.

Immune checkpoint inhibitors, including anti-PD-L1 antibodies, have been proposed for combination with many other types of anticancer therapies, including immunotherapies based on vaccines such as poxvirus vectors. In most cases, experiments have been conducted in animal models of cancer not involving HPV infection, and no specific analysis of toxicity of the tested combination has been performed (WO 2016/128542, WO 2015/175334, WO 2015/069571, Remy-Ziller et al., Hum Vaccin Immunother. (2018) 14(1): 140-145). Two phase II clinical trials (NCT03353675 and NCT02823990) combining TG4010 (an MVA vector encoding MUC1 and interleukin-2) and nivolumab (an anti-PD-1 antibody) in the treatment of non-small cell lung cancer (NSCLL) are ongoing (Oliveres H. et al., J Thorac Dis. (2018) 10(Suppl 13): S1602-S1614), but results of the trial have not been made public.

In the context of HPV-positive cancers, combinations of various vaccines intended to stimulate the immune response against HPV antigens, including viral vaccines, and of immune checkpoint inhibitors have also been proposed (Gildener-Leapman et al., Oral Oncol. (2014) 50(9): 780-4; WO 2015/103602; Rice et al., Cancer Gene Ther. (2015) 22(9): 454-62; WO 2016/071306; US 2017/051019; US 2019/142933). To date, there has been no disclosure of experimental data showing acceptable toxicity profile and improved therapeutic efficiency against HPV-positive cancer of a combination of a viral vaccine encoding HPV antigens and an anti-PD-L1 antibody. An ongoing phase Ib/II clinical trial (NCT03260023) combining TG4001 and avelumab in the treatment of HPV-positive SCCHN has been reported (Lin et al., Front Oncol. (2018) 8: 532), but results of the trial have not been made public.

### SUMMARY OF THE INVENTION

In the context of the present invention, the inventors surprisingly found that combining a poxvirus vector encoding at least human papillomavirus (HPV) E6 and E7 polypeptides and an immunostimulatory cytokine, preferably TG4001, and an anti-PD-L1 antibody or antigen-binding fragment thereof, preferably avelumab, using a specific administration scheme in HPV-positive cancer patients resulted in acceptable toxicities and improved immune response to HPV E6 and E7 polypeptides. In particular, despite the immune stimulating effect of both avelumab and TG4001 (live vector), no interaction between the two products leading to unacceptable toxicities was observed, which could not be reasonably expected. In particular, in view of the immune stimulating effects of both products, an interaction between both products that might lead to unacceptable toxicities was at risk, in particular in view of the fact that the US Food and Drug Administration (FDA) placed a clinical hold on a study combining a listeria vaccine with an anti-PD-L1 antibody after a patient died of complications of adverse events (NCT02291055). The case suggested that the vaccine (another type of live vector) may have amplified a known side effect of the immune checkpoint inhibitor. Moreover, promising beneficial immune changes were observed with the combination (including induction of immune response to HPV E6 and E7 polypeptides, increase in CD8 T cells and decrease of CD4 T regulatory cells in both circulation and tumor tissue, as well as upregulation of genes associated with a “hot” rather than “cold” tumor profile and with a better prognosis), which had never been observed with either treatment alone.

Potentiation may be additive, or it may be synergistic. The potentiating effect of the combination therapy is at least additive. The present inventors have surprisingly found that the combination of (a) a poxvirus vector encoding at least human papillomavirus (HPV) E6 and E7 polypeptides and an immunostimulatory cytokine and (b) an anti-PD-L1 antibody results in an improved treatment. Initial results in a clinical trial indicate that the combination therapy is effective at treating cancers such as recurrent/metastatic HPV16 positive cancers (see Example 1) and that the combination therapy is well tolerated (see Example 1). Moreover, effects attributable to each of the two treatments of the combination were observed (see Example 1), showing at least an additive potentiating effect of the combination therapy.

The present invention thus relates to a combination of:
a) a poxvirus vector encoding at least human papillomavirus (HPV) E6 and E7 polypeptides and an immunostimulatory cytokine, and
b) an anti-PD-L1 antibody or antigen-binding fragment thereof,

for use in the treatment of an HPV-positive cancer or HPV-positive precancerous intraepithelial lesions,

wherein a first administration of said poxvirus is performed 5 to 10 days before the first administration of said anti-PD-L1 antibody, and subsequent administrations of said poxvirus and anti-PD-L1 antibody are performed.

The present invention also relates to a method for treating an HPV-positive cancer or HPV-positive precancerous intraepithelial lesions in a subject in need thereof, comprising administering to said subject a combination of:
a) a poxvirus vector encoding at least human papillomavirus (HPV) E6 and E7 polypeptides and an immunostimulatory cytokine, and
b) an anti-PD-L1 antibody or antigen-binding fragment thereof,

wherein a first administration of said poxvirus is performed 5 to 10 days before the first administration of said anti-PD-L1 antibody, and subsequent administrations of said poxvirus and anti-PD-L1 antibody are performed.

The present invention also relates to the use of a combination of:
a) a poxvirus vector encoding at least human papillomavirus (HPV) E6 and E7 polypeptides and an immunostimulatory cytokine, and
b) an anti-PD-L1 antibody or antigen-binding fragment thereof,

for the manufacture of a medicament for use in the treatment of an HPV-positive cancer or HPV-positive precancerous intraepithelial lesions,

wherein a first administration of said poxvirus is performed 5 to 10 days before the first administration of said anti-PD-L1 antibody, and subsequent administrations of said poxvirus and anti-PD-L1 antibody are performed.

The present invention also relates to the use of a combination of:
a) a poxvirus vector encoding at least human papillomavirus (HPV) E6 and E7 polypeptides and an immunostimulatory cytokine, and
b) an anti-PD-L1 antibody or antigen-binding fragment thereof,

for the treatment of an HPV-positive cancer or HPV-positive precancerous intraepithelial lesions,

wherein a first administration of said poxvirus is performed 5 to 10 days before the first administration of said anti-PD-L1 antibody, and subsequent administrations of said poxvirus and anti-PD-L1 antibody are performed.

In said combination, method or use, said poxvirus is preferably a vaccinia virus, more preferably a modified Vaccinia Virus Ankara (MVA), and preferably encodes membrane anchored HPV (preferably HPV-16) non-oncogenic E6 and E7 polypeptides and human interleukin 2 (IL-2). Most preferably, said poxvirus is an MVA virus encoding membrane anchored HPV-16 non-oncogenic E6 and E7 polypeptides and human IL-2. Each dose of poxvirus administered to the subject is preferably of 3×10⁷ to 7×10⁷ pfu, more preferably about 5×10⁷ pfu. Each dose of poxvirus administered to the subject is preferably administered subcutaneously.

In said combination, method or use, said anti-PD-L1 antibody or antigen-binding fragment thereof preferably mediates antibody-dependent cell-mediated cytotoxicity (ADCC). Structurally, said anti-PD-L1 antibody or antigen-binding fragment thereof preferably comprises a heavy chain, which comprises three complementarity determining regions having amino acid sequences of SEQ ID Nos: 1, 2 and 3, and a light chain, which comprises three complementarity determining regions having amino acid sequences of SEQ ID Nos: 4, 5 and 6. More preferably, said anti-PD-L1 antibody comprises the heavy chain having amino acid sequences of SEQ ID NOs: 7 or 8 and the light chain having amino acid sequence of SEQ ID NO: 9. Most preferably, said anti-PD-L1 antibody is avelumab. Each dose of said anti-PD-L1 antibody or antigen-binding fragment thereof is preferably of about 10 mg/kg or about 800 mg. Each dose of said anti-PD-L1 antibody or antigen-binding fragment thereof is preferably administered intravenously, more preferably by intravenous infusion.

The targeted therapeutic use is the treatment of HPV-positive cancer, preferably of HPV-positive oropharyngeal, cervical, vaginal, anal, vulvar, penile, mucosal, or non-melanoma skin cancer, or of HPV-positive precancerous intraepithelial lesions. The cancer or the precancerous intraepithelial lesions is/are preferably HPV-16 positive, and the cancer may notably be HPV-16 positive squamous cell carcinoma of the head and neck (HPV-16+ SCCHN). The targeted cancer is further preferably a recurrent and/or metastatic HPV-positive cancer (preferably a recurrent and/or metastatic HPV16 positive cancer, most preferably recurrent and/or metastatic HPV16 positive SCCHN).

The combination of (a) a poxvirus vector encoding at least human papillomavirus (HPV) E6 and E7 polypeptides and an immunostimulatory cytokine, and (b) an anti-PD-L1 antibody can be provided in a single or separate unit dosage forms. The combination is administered according to a specific administration scheme, which comprises a first administration of said poxvirus 5 to 10 days before the first administration of said anti-PD-L1 antibody, and subsequent administrations of said poxvirus and anti-PD-L1 antibody. Preferably, said subsequent administrations of said poxvirus and anti-PD-L1 antibody are performed until disease progression. More preferably, the combination is administered with the following administration scheme:
a) a first dose of 3×10⁷ to 7×10⁷ pfu of said poxvirus is administered subcutaneously, and followed until disease progression by subsequent poxviruses doses of 3×10⁷ to 7×10⁷ pfu administered subcutaneously:
   on a weekly basis for 6 weeks,
   once every 2 weeks up to month 6, and
   every 12 weeks, for the next poxviruses doses;
b) a first dose of about 10 mg/kg or about 800 mg of anti-PD-L1 antibody is administered intravenously 5 to 10 days after the first poxvirus dose, and followed by subsequent anti-PD-L1 antibody doses of about 10 mg/kg or about 800 mg, administered intravenously every 2 weeks until disease progression.

A particularly preferred embodiment according to the invention is as follows:
a) Said poxvirus is an MVA virus encoding membrane anchored HPV-16 non-oncogenic E6 and E7 polypeptides and human IL-2,
b) Said anti-PD-L1 antibody is avelumab, and
c) Said poxvirus and anti-PD-L1 antibody are administered with the following administration scheme:
   i) The MVA virus encoding membrane anchored HPV-16 non-oncogenic E6 and E7 polypeptides and human IL-2 is administered subcutaneously at a dose of 5×10⁷ pfu on a weekly basis for 6 weeks, then once every 2 weeks up to Month 6, and every 12 weeks thereafter until disease progression,
   ii) Avelumab is administered by intravenous infusion at a dose of about or about 800 mg every 2 weeks starting from Day 8 until disease progression.

The combination, method or use according to the invention preferably induces positive immune responses against the treated subject's cancer or precancerous lesion. Notably, in circulation, the combination for use according to the invention preferably:
increases the proportion of circulating HPV E6 and/or E7 polypeptide-specific CD8 and/or CD4 T cells, and/or
decreases the proportion of circulating regulatory T cells.

Similarly, in tumor tissue, the combination, method or use according to the invention preferably:
increases the tumor tissue infiltration by HPV E6 and/or E7 polypeptide-specific CD8 and/or CD4 T cells, and/or
decreases the tumor tissue infiltration by regulatory T cells.

### DESCRIPTION OF THE FIGURES

FIG. 1. Changes in tumor size during combination treatment. (A) Best change in tumor size: % change from baseline (from calculated sum) at day 43 in individual patients treated by combination of TG4001 at DL1 (5×10⁶ pfu, plain grey) or DL2 (5×10⁷ pfu, hatched grey) with avelumab at 10 mg/kg according to the administration scheme described in Example 1. Partial response (PR) according to RECIST v1.1 is indicated by *(B). Individual longitudinal change: % change from baseline (from calculated sum) in individual patients treated by combination of TG4001 at DL1 (5×10⁶ pfu, dotted line) or DL2 (5×10⁷ pfu, plain line) with avelumab at 10 mg/kg according to the administration scheme described in Example 1 at various time points are presented. The threshold values for progressive disease (PD: ≥+20%), stable disease (−30%<SD<+20%), and partial response (PR: ≤−30%) are indicated. The type of HPV-16-positive cancer of each patient is also indicated.

FIG. 2. Stimulation of T cell immunity by the combination treatment. CD8/CD3 ratio (A) or Treg (CD4 Foxp3)/CD8 ratio (B) of tumor immune infiltrates at baseline and at day 43, based on immunohistochemistry (IHC) staining of tumor FFPE samples in individual patients.

FIG. 3. Analysis of gene expression change in tumor tissue during treatment. The expression of a panel of 770 genes related to immune response was assessed at baseline and after treatment (day 43). Volcano plots of changes in T cell activation (A), cytotoxic cells (B), pathogen defense (C) and NK cell function (D) gene expression post vs. pre-treatment. In each volcano plot, black dots correspond to genes of the indicated category.

FIG. 4. Analysis of gene expression change in tumor tissue during treatment. (A) Presentation of gene categories included in gene signatures previously described as Immunosign® 15 and Immunosign® 21 (Galon et al., Immunity (2013) 39(1): 11-26; Marabelle et al., Society for Immunotherapy of Cancer (SITC) 32^{nd} Annual Meeting [symbol] Pre-Conference Programs (SITC 2017) on Nov. 8-12, 2017 at the Gaylord National Hotel [symbol] Convention Center in National Harbor, Maryland. Poster P250). (B) Volcano plots of changes in Immunosign® 15 (B) and Immunosign® 21 (C) genes. In each volcano plot, black dots correspond to genes of the indicated signature.

FIG. 5. Changes in immune infiltrates in patient 0101006. (A) CD3, CD8, or CD4 Foxp3 T cells/mm² in tumor immune infiltrates at baseline and at day 43. (B). Percentage of CD8 T cells in the vicinity of PD-L1 expressing cells, depending on the distance in μm between PD-L1 expressing cells and CD8 T cells.

FIG. 6. Analysis of gene expression change in tumor tissue during treatment of patient 0101006. Expression of genes associated with antigen processing and presentation (A), genes associated with defense response to virus (B), of Toll-like receptors (C) and of Immunosign® 21 genes (D).

FIG. 7. Impact of disease/patient's characteristics on objective response rate (ORR). For each characteristic, the odds ratio (OR), 95% confidence interval and p-value is presented. Genital=vulvar/vaginal. An OR over 1 indicates that the presence of the characteristic is associated to a worse ORR, while an OR below 1 indicates that the presence of the characteristic is associated to a better ORR. A p-value <0.05 indicates that the characteristic is significantly associated to a worse or better ORR. Only one characteristic (presence of liver metastases) has been found significantly associated to a worse ORR (boxed).

FIG. 8. Impact of disease/patient's characteristics on progression free survival (PFS). For each characteristic, the hazard ratio (HR), 95% confidence interval and p-value is presented Genital=vulvar/vaginal. A HR over 1 indicates that the presence of the characteristic is associated to a worse PFS, while a HR below 1 indicates that the presence of the characteristic is associated to a better PFS. A p-value <0.05 indicates that the characteristic is significantly associated to a worse or better PFS. Two characteristics (presence of liver metastases and anal cancer) have been found significantly associated to a worse PFS, while one characteristic (lymph node involved) has been found significantly associated to a better PFS (all three characteristics are boxed).

FIG. 9. Best change in tumor size in 23 patients without liver metastasis from pooled phase Ib and phase II: best % change from baseline (from calculated sum) in individual patients treated by combination of TG4001 at DL2 (5×10⁷ pfu) with avelumab at 10 mg/kg according to the administration scheme described in Example 1. Progressive disease (PD) appears in black, stable disease (SD) in light grey, partial response (PR) in dark grey and complete response (CR) in medium grey.

FIG. 10. Best change in tumor size in 9 patients with liver metastasis from pooled phase Ib and phase II: best % change from baseline (from calculated sum) in individual patients treated by combination of TG4001 at DL2 (5×10⁷ pfu) with avelumab at 10 mg/kg according to the administration scheme described in Example 1. Progressive disease (PD) appears in black and stable disease (SD) in light grey.

FIG. 11. Volcano plot showing fold change for Yes vs No (Hepatic lesion) vs p value of Linear Model in Baseline (Visit) population. The most highly differentially expressed genes are named.

### DETAILED DESCRIPTION OF THE INVENTION

### General Definitions

The terms “a” and “an” are used herein throughout the entire application in the sense that they mean “at least one”, “at least a first”, “one or more” or “a plurality” of the referenced compounds or steps, unless the context dictates otherwise. The term thus includes both the case of only one of the referenced compounds or steps, and the case of more than one of the referenced compounds or steps.

The term “about” or “approximately” as used interchangeably herein means within 5%, preferably within 4%, and more preferably within 2% of a given value or range. In the context of the present disclosure, each time it is referred to an approximative value X by “about X”, the embodiment in which the value is equal to X is also contemplated in the context of the invention.

The term “and/or” wherever used herein includes the meaning of “and”, “or” and “all or any other combination of the elements connected by said term”. For example, “recurrent and/or metastatic” means recurrent, or metastatic or both recurrent and metastatic.

The term “combination” as used herein refers to any arrangement possible of two or more entities (e.g. at least the poxvirus and the anti-PD-L1 antibody described herein). In particular, a “combination” can refer to (i) a product comprised of two or more regulated components that are physically, chemically, or otherwise combined or mixed and produced as a single entity; (ii) two or more separate products packaged together in a single package or as a unit and comprised of drug and device products, device and biological products, or biological and drug products; (iii) a drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (iv) any investigational drug, device, or biological product packaged separately that according to its proposed labeling is for use only with another individually specified investigational drug, device, or biological product where both are required to achieve the intended use, indication, or effect.

The term “combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, three or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

The term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”), in each case as used herein, when used to define products, compositions and methods, are open-ended and do not exclude additional, unrecited elements or method steps. Thus, a polypeptide “comprises” an amino acid sequence when the amino acid sequence might be part of the final amino acid sequence of the polypeptide. “Consisting essentially of” shall mean excluding other components or steps of any essential significance. Thus, a composition consisting essentially of the recited components would not exclude trace contaminants and pharmaceutically acceptable carriers but would exclude other active ingredients. A polypeptide “consists essentially of” an amino acid sequence when such an amino acid sequence is present with optionally only a few additional amino acid residues. “Consisting of” means excluding more than trace elements of other components or steps. For example, a polypeptide “consists of” an amino acid sequence when the polypeptide does not contain any amino acids but the recited amino acid sequence. In the context of the present disclosure, each time a product or method or use is indicated as “comprising” something, the embodiment in which said product or method consists essentially of or consists of the same something is also contemplated in the context of the invention.

The term “mutant”, “analog” or “variant” as used herein refers to a component (polypeptide or nucleic acid) exhibiting one or more modification(s) with respect to its native counterpart. Any modification(s) can be envisaged, including substitution, insertion and/or deletion of one or more nucleotide/amino acid residue(s). When several mutations are contemplated, they can concern consecutive residues and/or non-consecutive residues. Mutation(s) can be generated by a number of ways known to those skilled in the art, such as site-directed mutagenesis (e.g., using the Sculptor™ in vitro mutagenesis system of Amersham, Les Ullis, France), PCR mutagenesis, DNA shuffling and by chemical synthetic techniques (e.g., resulting in a synthetic nucleic acid molecule). Preferred are analogs that retain a degree of sequence identity of at least 80%, preferably at least 85%, more preferably at least 90%, and even more preferably at least 91%, at least 92%, at least 93%, at least 94%, at least 95%, at least 96%, at least 97%, at least 98%, or at least 99% identity with the sequence of the native counterpart. In a general manner, the term “identity” refers to an amino acid to amino acid or nucleotide to nucleotide correspondence between two polypeptide or nucleic acid sequences. The percentage of identity between two sequences is a function of the number of identical positions shared by the sequences, taking into account the number of gaps which need to be introduced for optimal global alignment (i.e., optimal alignment of both full-length sequences) and the length of each gap. Various computer programs and mathematical algorithms are available in the art to determine the percentage of identity between amino acid or nucleic acid sequences.

The terms “nucleic acid”, “nucleic acid molecule”, “polynucleotide” and “nucleotide sequence” are used interchangeably and define a polymer of any length of either polydeoxyribonucleotides (DNA) (e.g. cDNA, genomic DNA, plasmids, vectors, viral genomes, isolated DNA, probes, primers and any mixture thereof) or polyribonucleotides (RNA) (e.g. mRNA, antisense RNA, SiRNA) or mixed polyribo-polydeoxyribonucleotides. They encompass single or double-stranded, linear or circular, natural or synthetic, modified or unmodified polynucleotides. Moreover, a polynucleotide may comprise non-naturally occurring nucleotides and may be interrupted by non-nucleotide components.

The terms “polypeptide”, “peptide” and “protein” refer to polymers of amino acid residues which comprise at least nine or more amino acids bonded via peptide bonds. The polymer can be linear, branched or cyclic and may comprise naturally occurring and/or amino acid analogs and it may be interrupted by non-amino acids. As a general indication, if the amino acid polymer is more than 50 amino acid residues, it is preferably referred to as a polypeptide or a protein whereas if it is 50 amino acids long or less, it is referred to as a “peptide”.

The term “subject” generally refers to an organism for whom any product and method of the invention is needed or may be beneficial. Typically, the organism is a mammal. Preferably, the subject is a human who has been diagnosed as being or at risk of having a pathological condition such as an infectious disease caused by or associated with a pathogenic organism or a proliferative disease such as cancer. The terms “subject” and “patient” may be used interchangeably when referring to a human organism and encompasses male and female. The subject to be treated may be a newborn, an infant, a young adult or an adult.

The terms “treatment” and “therapy”, as used in the present application, refer to a set of hygienic, pharmacological, surgical and/or physical means used with the intent to cure and/or alleviate a disease and/or the symptoms with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

### Poxvirus Vector

The combination, method or use according to the invention contains as first component a poxvirus vector encoding at least human papillomavirus (HPV) E6 and E7 polypeptides and an immunostimulatory cytokine.

The term terms “poxvirus vector” or “poxviral vector” have thus to be understood broadly as including a nucleic acid vector (e.g., DNA poxviral vector) that includes at least one element of a poxvirus genome and may be packaged into a poxviral particle as well as poxviral particles generated thereof. The terms “poxvirus”, “poxvirions”, “poxviral particles” and “poxviral vector particle” are used interchangeably to refer to poxviral particles that are formed when the nucleic acid vector is transduced into an appropriate cell or cell line according to suitable conditions allowing the generation of poxviral particles. The term “infectious” refers to the ability of a poxviral vector to infect and enter into a host cell or subject. Poxviral vectors can be replication-competent or replication-selective (e.g., engineered to replicate better or selectively in specific host cells), or can be genetically disabled to be replication-defective or replication-impaired.

### Type of Poxvirus

As used herein, the term “poxvirus” refers to a virus belonging to the Poxviridae family with a preference for the Chordopoxvirinae subfamily directed to vertebrate host, which includes several genera, such as Orthopoxvirus, Capripoxvirus, Avipoxvirus, Parapoxvirus, Leporipoxvirus and Suipoxvirus. Orthopoxviruses are preferred in the context of the present invention as well as the Avipoxviruses including Canarypoxvirus (e.g., ALVAC) and Fowlpoxvirus (e.g., the FP9 vector).

In a preferred embodiment of the combination, method or use according to the invention, the poxvirus belongs to the Orthopoxvirus genus and even more preferably to the vaccinia virus (VV) species. Vaccinia viruses are large, complex, enveloped viruses with a linear, double-stranded DNA genome of approximately 200 kb in length which encodes numerous viral enzymes and factors that enable the virus to replicate independently from the host cell machinery. Two distinct infectious viral particles exist, the intracellular IMV (for intracellular mature virion) surrounded by a single lipid envelop that remains in the cytosol of infected cells until lysis and the double enveloped EEV (for extracellular enveloped virion) that buds out from the infected cell.

A particularly appropriate poxvirus in the context of the present invention is MVA (Modified vaccinia virus Ankara) due to its highly attenuated phenotype, a more pronounced IFN-type 1 response generated upon infection compared to non-attenuated vectors and availability of the sequence of its genome (see e.g., Genbank under accession number U94848).

### HPV E6 and E7 Polypeptides

The poxvirus (preferably VV, more preferably MVA) of the combination, method or use according to the invention encodes at least human papillomavirus (HPV) E6 and E7 polypeptides and an immunostimulatory cytokine.

More than 100 HPV genotypes have been identified, which have been classified in “low” (LR) and “high risk” (HR) serotypes depending on their oncogenic potential. LR-HPV causes benign tumors in infected subjects whereas HR-HPV bears a high risk for malignant progression. The E6 and E7-encoded gene products of HR HPV genotypes are involved in the oncogenic transformation of infected cells, presumably through binding of these viral proteins to cellular tumor suppressor gene products p53 and retinoblastoma (Rb), respectively (reviewed in Howley, 1996, Papillomaviruses and their replication, p 2045-2076. In B. N. Fields, D. M. Knipe and P. M. Howley (ed), Virology, 3^{rd} ed. Lippincott-Raven Press, New York, N.Y.). The amino acid residues involved in the binding of the native HPV-16 E6 polypeptide to p53 have been clearly defined from residues 118 to 122 (+1 being the first Met residue or from residues 111 to 115 starting from the preferably used second Met residue) (Crook et al., Cell (1991) 67, 547-556) and those involved in the binding of the native HPV-16 E7 polypeptide to Rb are located from residues 21 to 26 (Heck et al., Proc. Natl. Acad. Sci. USA (1992) 89, 4442-4446).

In the context of the present invention, the poxvirus (preferably VV, more preferably MVA) preferably encodes at least E6 and E7 polypeptides of a HR-HPV, preferably selected from HPV-16, HPV-18, HPV-30, HPV-31, HPV-33, HPV-35, HPV-39, HPV-45, HPV-51, HPV-52, HPV-56, HPV-58, HPV-59, HPV-66, HPV-68, HPV-70 and HPV-85, more preferably from HPV-16 and HPV-18, and most preferably said HR-HPV is HPV-16.

Sources of papillomavirus include without limitation biological samples (e.g. biological samples, tissue sections, biopsy specimen and tissue cultures collected from a subject that has been exposed to a papillomavirus), cultured cells (e.g., CaSki cells available at ATCC), as well as recombinant materials available in depositary institutions, in commercial catalogues or described in the literature. The nucleotide sequences of a number of papillomavirus genomes and the amino acid sequences of the encoded polypeptides have been described in the literature and are available in specialized data banks, e.g., Genbank. For general information, the HPV-16 genome is described in Genbank under accession numbers NC_01526 and K02718; HPV-18 under NC_001357 and X05015; HPV-31 under J04353; HPV-33 under M12732; HPV-35 under NC_001529; HPV-39 under NC_001535; HPV-45 under X74479; HPV-51 under NC_001533; HPV-52 under NC_001592; HPV-56 under X74483; HPV-58 under D90400; HPV-59 under NC_001635; HPV-68 under X67160 and M73258; HPV-70 under U21941; and HPV-85 under AF131950.

For purpose of illustration, the amino acid sequences of native HPV-16 E6 and E7 polypeptides are given respectively in SEQ ID NOs: 10-11.

As defined above in connection with the term “polypeptide”, a “papillomavirus polypeptide” encompasses native, modified papillomavirus polypeptides and peptides thereof. In particular, the present invention encompasses the use/expression of native HPV E6 and E7 polypeptide(s) as well as analogs thereof (e.g., fragments thereof such as peptides; and modified ones), especially when the native polypeptide exerts undesired properties (e.g., oncogenic or transforming properties, cytotoxicity, etc). For example, to circumvent oncogenicity of HPV E6 and E7 polypeptides, one may use or express non-oncogenic analogs displaying reduced capacity to bind p53 and Rb, respectively.

Suitable E6 polypeptides for use in the invention encompass non-oncogenic mutants that are defective in binding to the cellular tumor suppressor gene product p53. Representative examples of non-oncogenic E6 polypeptides are described in the art (see e.g., WO 1999/03885). Preferred modifications in this context include the deletion in HPV-16 E6 of one or more amino acid residues located from approximately position 118 to approximately position 122 (+1 representing the first methionine residue of the native HPV-16 E6 polypeptide), with a special preference for the deletion in HPV-16 E6 of residues 118 to 122 (CPEEK) (see e.g., SEQ ID NO: 12) or the deletion in HPV-18 E6 of residues 113 to 117 (NPAEK).

Suitable E7 polypeptides for use in the invention encompass non-oncogenic mutants that are defective in binding to the cellular tumor suppressor gene product Rb. Representative examples of non-oncogenic E7 polypeptides are described in the art (see e.g., WO 1999/03885). Preferred modifications in this context include the deletion in HPV-16 E7 of one or more amino acid residues located from approximately position 21 to approximately position 26 (+1 representing the first amino acid of the native HPV-16 E7 polypeptide, with a special preference for the deletion in HPV-16 E7 of residues 21 to 26 (DLYCYE) (see e.g., SEQ ID NO: 13) or the deletion in HPV-18 E7 of residues 24 to 28 (DLLCH).

The HPV (preferably HPV-16) E6 and/or E7 polypeptides for use in the invention may further have been modified to be membrane anchored, enhancing efficient membrane presentation of the polypeptide(s) at the surface of the expressing host cell. This may be achieved by fusing the HPV (preferably HPV-16) E6 and/or E7 polypeptide(s) to a signal peptide and a membrane-anchoring peptide. Such peptides are known in the art. Briefly, signal peptides are generally present at the N-terminus of membrane-presented or secreted polypeptides and initiate their passage into the endoplasmic reticulum (ER). They comprise 15 to 35 essentially hydrophobic amino acids which are then removed by a specific ER-located endopeptidase to give the mature polypeptide. Membrane-anchoring peptides are usually highly hydrophobic in nature and serve to anchor the polypeptides in the cell membrane (see for example Branden and Tooze, 1991, in Introduction to Protein Structure p. 202-214, NY Garland). The choice of the signal and membrane-anchoring peptides which can be used in the context of the present invention is vast. They may be independently obtained from any secreted or membrane-anchored polypeptide (e.g. cellular or viral polypeptides) such as the rabies glycoprotein, the HIV virus envelope glycoprotein or the measles virus F protein or may be synthetic. The preferred site of insertion of the signal peptide is the N-terminus downstream of the codon for initiation of translation and that of the membrane-anchoring peptide is the C-terminus, for example immediately upstream of the stop codon. If necessary, a linker peptide can be used to connect the signal peptide and/or the membrane anchoring peptide to the encoded polypeptide.

The poxvirus of the combination treatment according to the invention preferably encodes HPV (preferably HPV-16) membrane anchored and non-oncogenic E6 and E7 polypeptides and human interleukin 2 (IL-2).

In a particularly preferred embodiment, the HPV E6 polypeptide encoded by the poxvirus (preferably VV, more preferably MVA) of the combination for use according to the invention is a membrane-anchored and non-oncogenic variant of HPV-16 E6 with a deletion in HPV-16 E6 of residues 118 to 122 (CPEEK), especially the HPV-16 E6 variant of amino acid sequence SEQ ID NO: 12. In another particularly preferred embodiment, the HPV E7 polypeptide encoded by the poxvirus (preferably VV, more preferably MVA) of the combination for use according to the invention is a membrane-anchored and non-oncogenic variant of HPV-16 E7 with a deletion in HPV-16 E7 of residues 21 to 26 (DLYCYE), especially the HPV-16 E7 variant of amino acid sequence SEQ ID NO: 13. In a particularly preferred embodiment, the HPV E6 polypeptide encoded by the poxvirus (preferably VV, more preferably MVA) of the combination for use according to the invention is a membrane-anchored and non-oncogenic variant of HPV-16 E6 with a deletion in HPV-16 E6 of residues 118 to 122 (CPEEK), especially the HPV-16 E6 variant of amino acid sequence SEQ ID NO: 12, and the HPV E7 polypeptide encoded by the poxvirus (preferably VV, more preferably MVA) of the combination for use according to the invention is a membrane-anchored and non-oncogenic variant of HPV-16 E7 with a deletion in HPV-16 E7 of residues 21 to 26 (DLYCYE), especially the HPV-16 E7 variant of amino acid sequence SEQ ID NO: 13.

Suitable promoters for driving expression of the at least human papillomavirus (HPV) E6 and E7 polypeptides and immunostimulatory cytokine encoded by the poxvirus vector comprised in the combination, method or use according to the invention are preferably poxviral promoters, for example vaccinia virus promoters 7.5K, H5R, TK, p.28, p.11 or K1L. Synthetic promoters are also suitable as well as chimeric promoters between a late promoter and an early promoter. Such promoters are well known in the art. Preferably, expression of both HPV E6 and E7 polypeptides is placed under the control of the vaccinia p7.5 promoter and expression of the immunostimulatory cytokine (e.g. human IL-2) under the control of the vaccinia pH5R promoter.

### Immunostimulatory Cytokine

In addition to at least human papillomavirus (HPV) E6 and E7 polypeptides (preferably those described above), the poxvirus (preferably VV, more preferably MVA) of the combination, method or use according to the invention further encodes an immunostimulatory cytokine.

As used herein, the term “immunostimulatory cytokine” refers to a cytokine which has the ability to stimulate the immune system, in a specific or non-specific way. A vast number of cytokines are known in the art for their ability to exert an immunostimulatory effect. Examples of suitable immunostimulatory cytokines in the context of the invention include, without limitation, interleukins (e.g., IL-2, IL-6, IL-12, IL-15, IL-24), chemokines (e.g., CXCL10, CXCL9, CXCL11), interferons (e.g., IFNα, IFNβ, IFNγ), tumor necrosis factor (TNF), colony-stimulating factors (e.g. GM-CSF, C-CSF, M-CSF), growth factors (Transforming Growth Factor TGF, Fibroblast Growth Factor FGF, Vascular Endothelial Growth Factors VEGF, and the like). Preferably, the immunostimulatory cytokine is an interleukin or a colony-stimulating factor (e.g., GM-CSF). More preferably, the immunostimulatory cytokine is interleukin 2 (IL-2), most preferably human IL-2.

### Preferred Poxviruses

A preferred poxvirus of the combination, method or use according to the invention is an MVA virus encoding membrane-anchored non-oncogenic HPV-16 E6 and E7 polypeptides and human IL-2, more preferably represented by TG4001, as described in WO 1999/03885 under its research name MVATG8042.

### Anti-PD-L1 Antibody or Antigen-Binding Fragment Thereof

The combination, method or use according to the invention contains as second component an anti-PD-L1 antibody or antigen-binding fragment thereof.

### Antibody or Antigen-Binding Fragment Thereof

The term “antibody” refers to an immunoglobulin molecule capable of specific binding to an antigen, such as a carbohydrate, polynucleotide, lipid, polypeptide, etc., through at least one antigen recognition site, located in the variable region of the immunoglobulin molecule. In the context of the invention, “antibody” (or “Ab”) is used in the broadest sense and encompasses naturally occurring antibodies as well as those engineered by man, including full length antibodies or functional fragments or analogs thereof that are capable of binding an antigen, such as PD-L1 (thus retaining the antigen-binding portion). The antibody in use in the invention can be of any origin, e.g., human, humanized, animal (e.g., rodent or camelid antibody) or chimeric. It may be of any isotype (e.g., IgG1, IgG2, IgG3, IgG4, IgM, etc.). In addition, it may be glycosylated or non-glycosylated. The term antibody also includes bispecific or multispecific antibodies so long as they exhibit binding specificity for an antigen, such as PD-L1. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates), any other modified configuration of the immunoglobulin molecule that comprises an antigen recognition site, antibody compositions with poly-epitopic specificity, and multi-specific antibodies (e.g., bispecific antibodies). However, intact, i.e., non-fragmented, monoclonal antibodies are preferred.

For illustrative purposes, full length antibodies are glycoproteins comprising two heavy (H) chains and two light (L) chains inter-connected by disulfide bonds. Each heavy chain is comprised of a heavy chain variable region (VH) and a heavy chain constant region (CH), which is made of three CH1, CH2 and CH3 domains (optionally with a hinge between CH1 and CH2). Each light chain is comprised of a light chain variable region (VL) and a light chain constant region, which comprises one CL domain. Each VH and VL region comprises three hypervariable regions, named complementarity determining regions (CDR), and interspersed with more conserved regions named framework regions (FR). Each VH and VL is composed of three CDRs and four FRs in the following order: FR1-CDR1-FR2-CDR2-FR3-CDR3-FR4. The CDR regions of the heavy and light chains are generally determinant for the binding specificity.

An antibody's CDRs are defined by the amino acid sequence of its heavy and light chains compared to criteria known to a person skilled in the art. Various methods for determining CDRs have been proposed, and the portion of the amino acid sequence of a heavy or light chain variable region of an antibody defined as a CDR varies according to the method chosen. In the present description, all CDRs are defined in accord with the AbM definition used by Oxford Molecular's AbM antibody modeling software (see e.g., CDR sequences of avelumab in WO 2013/079174).

The antibody may be a monoclonal antibody, human antibody, chimeric, humanized antibody and/or human antibody, and may include a human constant region. Constant regions of the antibodies can be altered, e.g., mutated, to modify the properties of the antibody (e.g., to increase or decrease one or more of: Fc receptor binding, antibody glycosylation, the number of cysteine residues, effector cell function or complement function).

In the context of the invention, a monoclonal antibody is preferably used. As used herein, a “monoclonal antibody” refers to a composition comprising antibody molecules having an identical and unique antigen specificity. The antibody molecules present in the composition are likely to vary in terms of their post-translational modifications, and notably in terms of their glycosylation structures or their isoelectric point but have all been encoded by the same heavy and light chain sequences and thus have, before any post-translational modification, the same protein sequence. Certain differences in protein sequences, related to post-translational modifications (such as for example cleavage of the heavy chain C-terminal lysine, deamidation of asparagine residues and/or isomerization of aspartate residues), may nevertheless exist between the various antibody molecules present in the composition. A monoclonal antibody can be made by hybridoma technology or by methods that do not use hybridoma technology (e.g., recombinant methods). Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein.

For the treatment of human subjects, which is a preferred embodiment of the invention, the anti-PD-L1 antibody will preferably be chimeric, humanized or fully human, thus limiting or preventing immune responses directed against non-human parts of the anti-PD-L1 antibody. An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

As used herein, a “chimeric antibody” refers to an antibody comprising one or more element(s) of one species and one or more element(s) of another species, for example, a non-human antibody comprising at least a portion of a constant region (Fc) of a human immunoglobulin. Chimeric antibodies can be produced by recombinant DNA techniques known in the art.

As used herein, a “humanized antibody” refers to a non-human (e.g., murine, camel, rat, etc.) antibody whose protein sequence has been modified to increase its similarity to a human antibody (i.e. produced naturally in humans). An antibody can be humanized by methods known in the art. For example, a monoclonal antibody developed for human use can be humanized by substituting one or more residue of the FR regions to look like human immunoglobulin sequence whereas the vast majority of the residues of the variable regions (especially the CDRs) are not modified and correspond to those of a non-human immunoglobulin. For general guidance, the number of these amino acid substitutions in the FR regions is typically no more than 20 in each variable region VH or VL. In another example, a humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to anti-PD-L1. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor”. In one embodiment, the donor immunoglobulin is a non-human (e.g., rodent) immunoglobulin. The acceptor framework is a naturally-occurring (e.g., a human) framework or a consensus framework, or a sequence about 85% or higher, preferably 90%, 95%, 99% or higher identical thereto. Humanized or CDR-grafted antibodies can be produced by CDR-grafting or CDR substitution, wherein one, two, or all CDRs of an immunoglobulin chain can be replaced. See e.g., U.S. Pat. No. 5,225,539, which describes a CDR-grafting method which may be used to prepare the humanized antibodies of the present invention.

As used herein, a “human antibody” refers to an antibody, in which not only the constant (as in chimeric antibodies) and FR (as in humanized antibodies) regions are of human origin, but the whole amino acid sequences of the heavy and light chains are derived from human germline immunoglobin sequences. Such human antibodies may, for instance, be obtained from transgenic animals, in which human germline immunoglobin sequences have been inserted or from human antibody libraries.

The term “antigen-binding fragment” of any antibody refers to a portion of an intact antibody that binds to an antigen. An antigen-binding fragment can contain the antigenic determining variable regions of an intact antibody. The antigen-binding fragment can be engineered for use in the combination of the invention. Representative examples include without limitation Fab, Fab′, F(ab′)2, dAb, Fd, Fv, scFv, di-scFv, diabody and any other artificial antibody. For example, “PD-L1-binding fragment” of any anti-PD-L1 antibody refers to a portion of an intact antibody that binds to the antigen PD-L1. More specifically, the following antigen-binding fragments of a full-length anti-PD-L1 antibody may be used in the combination, method or use according to the invention:
(i) a Fab fragment is represented by a monovalent fragment consisting of the VL, VH, CL and CH1 domains;
(ii) a F(ab′)2 fragment is represented by a bivalent fragment comprising two Fab fragments linked by at least one disulfide bridge at the hinge region;
(iii) a Fd fragment consists of the VH and CH1 domains;
(iv) a Fv fragment consists of the VL and VH domains of a single arm of an antibody,
(v) a dAb fragment consists of a single variable domain fragment (VH or VL domain);
(vi) a single chain Fv (scFv) comprises the two domains of a Fv fragment, VL and VH, that are fused together, optionally with a linker to make a single protein chain; and
(vii) any other artificial antibody.

Methods for preparing antibodies, fragments and analogs thereof are known in the art (see e.g. Harlow and Lane, 1988, Antibodies—A laboratory manual; Cold Spring Harbor Laboratory, Cold Spring Harbor NY). In one embodiment, such an antibody can be generated in a host animal with a PD-L1 antigen (preferably a human PD-L1 antigen for human use). Alternatively, it can be produced from hybridomas (see e.g., Kohler and Milstein, Nature (1975) 256: 495-7), recombinant techniques (e.g. using phage display methods), peptide synthesis and enzymatic cleavage. Antibody fragments can be produced by recombinant technique as described herein. They may also be produced by proteolytic cleavage with enzymes such as papain to produce Fab fragments or pepsin to produce F(ab′)2 fragments. Analogs (or fragment thereof) can be generated by conventional molecular biology methods (PCR, mutagenesis techniques). If needed, such fragments and analogs may be screened for functionality in the same manner as intact antibodies (e.g. by standard ELISA assay).

### PD-1 and PD-L1

Programmed Death 1 (PD-1) is part of the immunoglobulin (Ig) gene superfamily and a member of the CD28 family. It is a 55 kDa type 1 transmembrane protein expressed on antigen-experienced cells (e.g., activated B cells, T cells, and myeloid cells). In normal context, it acts by limiting the activity of T cells at the time of inflammatory response, thereby protecting normal tissues from destruction. Two ligands have been identified for PD-1, respectively PD-L1 (programmed death ligand 1) and PD-L2 (programmed death ligand 2). PD-L1 was identified in 20-50% of human cancers. The interaction between PD-1 and PD-L1 resulted in a decrease in tumor infiltrating lymphocytes, a decrease in T-cell receptor mediated proliferation, and immune evasion by the cancerous cells. A full-length amino acid sequence for PD-1 is provided in UniProtKB under accession no. Q15116.

The anti-PD-L1 antibody of the combination, method or use according to the invention preferably recognizes human PD-L1, for which additional information (including known amino acid sequences) is also available in UniProtKB database under accession number Q9NZQ7.

### Functional Features of the Anti-PD-L1 Antibody

The term “anti-PD-L1 antibody” refers to an antibody that is capable of specifically binding PD-L1 with sufficient affinity such that the antibody blocks binding of PD-L1 to PD-1 and is thus useful as a therapeutic agent in targeting PD-L1 (e.g., avelumab). In particular, an anti-PD-L1 antibody means an antibody that blocks binding of PD-L1 expressed on a cancer cell to PD-1.

The term “antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils, and macrophages) enable these cytotoxic effector cells to bind specifically to an antigen-bearing target cell and subsequently kill the target cell with cytotoxins. The antibodies arm the cytotoxic cells and are required for killing of the target cell by this mechanism. The primary cells for mediating ADCC, the NK cells, express FcγRIII only, whereas monocytes express FcγRI, FcγRII and FcγRIII. Fc expression on hematopoietic cells is summarized in Table 3 on page 464 of Ravetch [symbol] Kinet, Annu Rev Immunol (1991) 9: 457-92. Thus, an anti-PD-L1 antibody, which comprises an ADCC-competent Fc region, may improve the efficacy of the present therapy by promoting ADCC lysis of the cancer cells. The anti-PD-L1 antibody of the combination, method or use according to the invention thus preferably mediates ADCC. In particular, a full-length antibody comprising a functional Fc region is preferably used. The Fc region may possibly be modified (at the amino acid or glycosylation level) in order to further improve ADCC ability (such modifications notably include one or more substitutions in the Fc and/or reduced fucosylation, which are well known in the art). Nevertheless, such ADCC-mediating anti-PD-L1 antibody is not toxic or does not show increased toxicity.

### Structural Features of the Anti-PD-L1 Antibody

Examples of monoclonal antibodies that bind to human PD-L1, and useful in the combination for use of the present invention, are described in WO 2007/005874, WO 2010/036959, WO 2010/077634, WO 2010/089411, WO 2013/019906, WO 2013/079174, WO 2014/100079, WO 2015/061668, and U.S. Pat. Nos. 8,552,154, 8,779,108 and 8,383,796. Specific anti-human PD-L1 monoclonal antibodies useful as the PD-L1 antibody in the combination for use of the present invention include, for example without limitation, avelumab (MSB0010718C), durvalumab (MEDI4736, an engineered IgG1 kappa monoclonal antibody with triple mutations in the Fc domain to remove ADCC), atezolizumab (MPLDL3280A), MPDL3280A (an IgG1-engineered anti-PD-L1 antibody), and BMS-936559 (a fully human, anti-PD-L1, IgG4 monoclonal antibody).

Avelumab and atezolizumab are unique among currently employed anti-PD-L1 antibodies in that they are fully human IgGs with a non-mutated Fc region. Thus, avelumab comprises an antibody-dependent cellular cytotoxicity (ADCC) competent Fc region which has been shown to mediate ADCC (Boyerinas et al., Cancer Immunol Res. (2015) 3(10):1148-1157). An antibody which comprises an ADCC-competent Fc region may improve the efficacy of the present therapy by promoting ADCC lysis of the cancer cells.

In an embodiment, the anti-PD-L1 antibody or antigen-binding fragment thereof comprises a heavy chain, which comprises three complementarity determining regions having amino acid sequences of SEQ ID NO:1 (avelumab H-CDR1: SYIMM), SEQ ID NO:2 (avelumab H-CDR2: SIYPSGGITFYADTVKG) and SEQ ID NO:3 (avelumab H-CDR3: IKLGTVTTVDY), and a light chain, which comprises three complementarity determining regions having amino acid sequences of SEQ ID NO:4 (avelumab L-CDR1: TGTSSDVGGYNYVS), SEQ ID NO:5 (avelumab L-CDR2: DVSNRPS) and SEQ ID NO:6 (avelumab L-CDR3: SSYTSSSTRV). Since CDR regions are known to be particularly involved in antigen recognition, such anti-PD-L1 antibody or antigen-binding fragment thereof is expected to have similar binding to PD-L1 as avelumab.

It is frequently observed that in the course of antibody production the C-terminal lysine (K) of the heavy chain is cleaved off. This modification has no influence on the antibody-antigen binding. Therefore, in some preferred embodiments, the anti-PD-L1 antibody comprises the heavy chain having the amino acid sequence of SEQ ID NOs: 7, in which the C-terminal lysine (K) is absent (avelumab heavy chain: EVQLLESGGGLVQPGGSLRLSCAASGFTFSSYIMMWVRQAPGKGLEWVSSIYPSGGITFYADTVKGRFTI SRDNSKNTLYLQMNSLRAEDTAVYYCARIKLGTVTTVDYWGQGTLVTVSSASTKGPSVFPLAPSSKSTS GGTAALGCLVKDYFPEPVTVSWNSGALTSGVHTFPAVLQSSGLYSLSSVVTVPSSSLGTQTYICNVNHK PSNTKVDKKVEPKSCDKTHTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCKVSNKALPAPIEKTISKAKGQP REPQVYTLPPSRDELTKNQVSLTCLVKGFYPSDIAVEWESNGQPENNYKTTPPVLDSDGSFFLYSKLTVD KSRWQQGNVFSCSVMHEALHNHYTQKSLSLSPG) or SEQ ID NO: 8, in which lysine (K) is present (VQLLESGGGLVQPGGSLRLSCAASGFTFSSYIMMWVRQAPGKGLEWVSSIYPSGGITFYADTVKGRFTI SRDNSKNTLYLQMNSLRAEDTAVYYCARIKLGTVTTVDYWGQGTLVTVSSASTKGPSVFPLAPSSKSTS GGTAALGCLVKDYFPEPVTVSWNSGALTSGVHTFPAVLQSSGLYSLSSVVTVPSSSLGTQTYICNVNHK PSNTKVDKKVEPKSCDKTHTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCKVSNKALPAPIEKTISKAKGQP REPQVYTLPPSRDELTKNQVSLTCLVKGFYPSDIAVEWESNGQPENNYKTTPPVLDSDGSFFLYSKLTVD KSRWQQGNVFSCSVMHEALHNHYTQKSLSLSPGK), and the light chain having amino acid sequence of SEQ ID NO: 9 (avelumab heavy chain: QSALTQPASVSGSPGQSITISCTGTSSDVGGYNYVSWYQQHPGKAPKLMIYDVSNRPSGVSNRFSGSKS GNTASLTISGLQAEDEADYYCSSYTSSSTRVFGTGTKVTVLGQPKANPTVTLFPPSSEELQANKATLVCLI SDFYPGAVTVAWKADGSPVKAGVETTKPSKQSNNKYAASSYLSLTPEQWKSHRSYSCQVTHEGSTVEK TVAPTECS).

### Preferred Anti-PD-L1 Antibodies

Preferably, the anti-PD-L1 antibody of the combination for use according to the invention is avelumab or an antibody or antigen-binding fragment thereof with structural similarity to avelumab. Avelumab, its sequence, and many of its properties have been described in WO 2013/079174, where it is designated A09-246-2 having the heavy and light chain sequences according to SEQ ID NOs: 32 and 33 (corresponding herein to SEQ ID NOs: 7 and 9). Avelumab has two main mechanisms of action for exerting its anti-tumor effects: First, PD-L1 on tumor cells can interact with PD-1 or B7-1 on activated T cells. These interactions have been shown to significantly inhibit T cell activities. Therefore, blocking PD-L1 interaction with PD-1 or B7-1 by anti-PD-L1 can release T cells from immunosuppression, and lead to elimination of tumor cells by T cells. Secondly, tumor cells may express high levels of PD-L1 on their surface compared with normal tissues. As a fully human IgG1 monoclonal antibody, avelumab has ADCC potential. Upon binding to PD-L1 on tumor cells and binding with their Fc part to Fc-gamma receptors on leukocytes, avelumab can trigger tumor-directed ADCC.

In a preferred embodiment of the invention, the anti-PD-L1 antibody comprises the 6 CDRs of avelumab (SEQ ID NOs: 1 to 6) and blocks the interaction between human PD-1 and human PD-L1. Preferably, said anti-PD-L1 antibody further mediates ADCC. More preferably, said anti-PD-L1 antibody is an IgG antibody, with a specific preference for an IgG1 antibody.

In a more preferred embodiment of the invention, the anti-PD-L1 antibody comprises the amino acid sequences of the heavy (SEQ ID NOs: 7 or 8) and light (SEQ ID NO: 9) chains of avelumab and blocks the interaction between human PD-1 and human PD-L1. Preferably, said anti-PD-L1 antibody further mediates ADCC. More preferably, said anti-PD-L1 antibody is an IgG antibody, with a specific preference for an IgG1 antibody.

In a most preferred embodiment of the invention, the anti-PD-L1 antibody is avelumab.

### Cancer or Precancerous Lesions to be Treated

The term “cancer” refers to a group of diseases, which can be defined as any abnormal malignant new growth of tissue that possesses no physiological function and arises from uncontrolled usually rapid cellular proliferation and has the potential to invade or spread to other parts of the body. The term “precancerous lesion” refers to a benign lesion involving abnormal cells, which are associated with an increased risk of developing into cancer.

In one aspect of the invention, the targeted therapeutic use is the treatment of HPV-positive cancer or precancerous intraepithelial lesions.

As used herein, an “HPV-positive cancer” and “HPV-positive precancerous intraepithelial lesions” respectively refer to a cancer or precancerous intraepithelial lesions caused or associated with HPV infection, in which the presence of HPV virus may be detected.

HR-HPVs produce 2 oncoproteins, E6 and E7, which are necessary for viral replication through their proliferation stimulating activity and play a key role in malignant transformation. The E6 oncoprotein binds and induces the degradation of the p53 tumor suppressor protein via an ubiquitin-mediated process disrupting the p53 pathway which leads to uncontrolled cell cycle progression. The HPV E7 protein binds and degrades the retinoblastoma protein (pRb), preventing it from inhibiting the transcription factor E2F resulting in loss of cell cycle control. Furthermore, the functional inactivation of Rb results in upregulation of the p16-protein. P16 is encoded by the CDKN2A tumor suppressor gene and regulates the activity of Cyclin D-CDK4/6 complexes that phosphorylate Rb leading to release of the transcription factor E2F which initiates cell cycle progression. HPV-positive tumors are characterized by high expression of high levels of p16 (Nevins J. R., Hum Mol Genet. (2001) 10(7): 699-703).

The presence of HPV virus may be detected by various methods, based on detection of HPV DNA, HPV RNA, HPV oncoproteins, or indirectly by searching for altered expression of cellular proteins such as overexpression of the p16 protein. The p16 protein can be detected by immunohistochemistry (IHC), and since several studies have shown a very high correlation (>90%) to HPV-positivity in oropharyngeal tumors, it has been suggested as a clinically useful surrogate marker (Mellin Dahlstrand H. et al., Anticancer Res. (2005) 25(6C): 4375-4383. Presence of HPV (and thus the HPV-positive nature of the cancer or precancerous intraepithelial lesions) may also be determined by detecting (1) HPV DNA, (2) post-integration transcription of viral E6 and/or E7 mRNA, (3) the viral oncoproteins E6 and E7, or (4) altered expression of cellular proteins such as overexpression of the p16 protein (Kim et al., J Pathol Clin Res. (2018) 4(4): 213-226). HPV DNA may notably be detected using polymerase chain reaction (PCR) or in situ hybridization (ISH). HPV RNA may notably be detected using polymerase chain reaction (RT-PCR) or in situ hybridization (ISH). Various kits for HPV status (positive or negative) determination of cancerous or precancerous lesions are commercially available and may be used in the context of the present invention (see Table 1 of Kim et al., J Pathol Clin Res. (2018) 4(4): 213-226).

In a preferred embodiment, since HPV-16 is the main HR-HPV detected in HPV-positive cancers, HPV status of cancerous or precancerous lesions is determined by detecting HPV-16 E7 DNA by PCR using HPV-16 specific primers. In a more preferred embodiment, DNA is extracted from a tumor sample (e.g., fixated tumor sample, such as a formol or formalin-fixed paraffin-embedded (FFPE) tumor sample) of the subject to be treated by conventional methods, HPV-16 E7 DNA is then amplified by PCR using HPV-16 specific primers. If amplification is detected (e.g., by immunofluorescent means such as SYBRgreen or others), then the sample is considered as HPV-16 positive. If amplification is not detected by this method, then HPV E7 DNA is amplified by PCR using consensus primers able to amplify about 50 HPV genotypes, the amplified sequences are then sequenced using Sanger sequencing. The obtained sequences then make it possible to confirm the negativity, to identify patients in whom the quality of the sample did not make it possible to obtain the result with the first-line PCR or to detect positive patients with rarer genotypes.

Preferred HPV-positive cancers include HPV-positive oropharyngeal, cervical, vaginal, anal, vulvar, penile, mucosal, or non-melanoma skin cancer. Among HPV-positive oropharyngeal cancers, squamous cell carcinoma of the head and neck (SCCHN) is preferred.

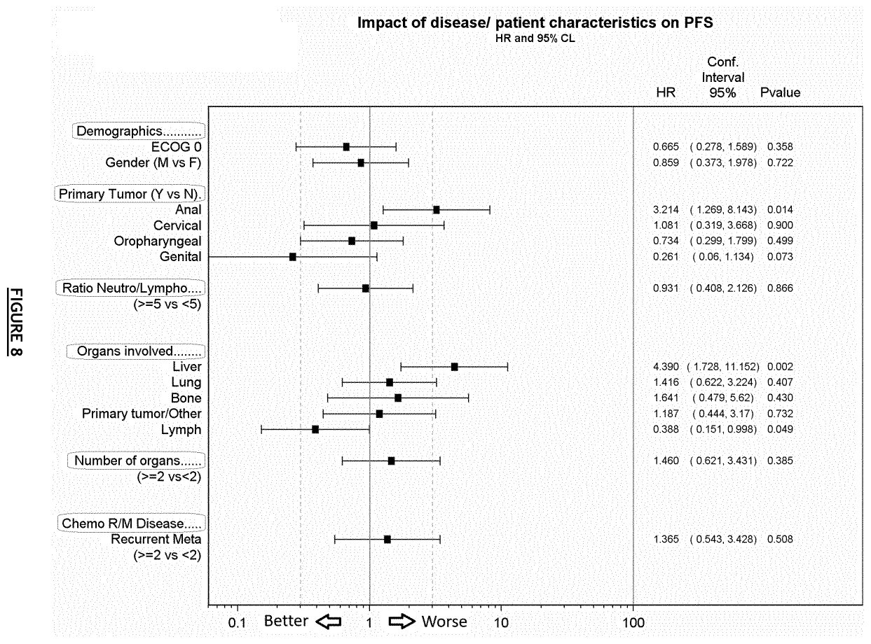

In their pooled interim analysis of phase Ib and phase II clinical trials, the inventors showed that HPV-positive anal cancer is significantly associated to lower progression-free survival (PFS, see FIG. 8), which is in fact due to a higher prevalence of liver metastasis in anal cancer patients, and not to anal cancer itself. Indeed, some patients with anal cancer but no liver metastasis (but other metastasis) reply to the treatment. Therefore, among HPV-positive cancers, and in particular those listed above, the HPV-positive cancer is preferably not HPV-positive anal cancer due to its higher prevalence of liver metastasis, and in particular not anal cancer with liver metastasis, and is thus preferably selected from HPV-positive oropharyngeal (in particular SCCHN), cervical, vaginal, vulvar, penile, mucosal, or non-melanoma skin cancer.

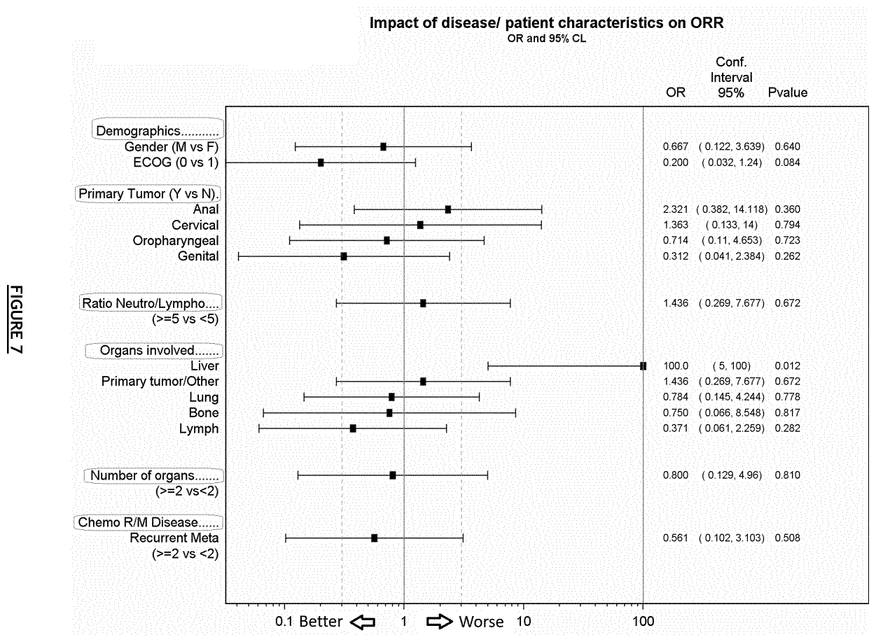

In contrast, the inventors also observed a high response rate in the HPV-positive vulvar/vaginal cancer patients (see Table 11), as well as non-significant tendency of association of the HPV-positive genital (meaning vulvar/vaginal) cancer with better objective response rate (ORR, see FIG. 7) and PFS (see FIG. 8). Therefore, among HPV-positive cancers, HPV-positive vulvar and vaginal cancers are preferred.

Preferred HPV-positive precancerous intraepithelial lesions include cervical intraepithelial neoplasia (CIN) grade 2 or 3 and vulvar intraepithelial neoplasia (VIN) grade 2 or 3. Cervical intraepithelial neoplasia (CIN) is a premalignant lesion that may exist at any one of three stages: CIN1, CIN2, or CIN3. If left untreated, CIN2 or CIN3 (collectively referred to as CIN2+) can progress to cervical cancer. Similarly, vulvar intraepithelial neoplasia (VIN) is a premalignant lesion that may exist at any one of three stages: VIN1, VIN2, or VIN3. If left untreated, VIN2 or VIN3 (collectively referred to as VIN2+) can progress to vulvar cancer.

The cancer or the precancerous intraepithelial lesions to be treated is/are preferably positive for a HR-HPV, which preferably corresponds to the HR-HPV which the HPV E6 and E7 polypeptides encoded by the poxvirus originate from. The cancer or the precancerous intraepithelial lesions to be treated is/are preferably positive for a HR-HPV selected from HPV-16, HPV-18, HPV-30, HPV-31, HPV-33, HPV-35, HPV-39, HPV-45, HPV-51, HPV-52, HPV-56, HPV-58, HPV-59, HPV-66, HPV-68, HPV-70 and HPV-85, more preferably from HPV-16 and HPV-18, and most preferably the cancer or the precancerous intraepithelial lesions to be treated is/are positive for HPV16. When the cancer or the precancerous intraepithelial lesions to be treated is/are positive for HPV-16, the poxvirus preferably encodes HPV-16 E6 and E7 polypeptides (more preferably non-oncogenic versions thereof, as disclosed above).

In a preferred embodiment, the targeted therapeutic use is thus an HPV-16 positive cancer (preferably not HPV16-positive anal cancer with liver metastasis, or more generally not HPV16-positive anal cancer, due to its high prevalence of liver metastasis) or HPV-16 positive precancerous intraepithelial lesions, and the cancer may notably be selected from HPV16-positive oropharyngeal (in particular SCCHN), cervical, vaginal, vulvar, penile, mucosal, or non-melanoma skin cancer. In preferred embodiments, the cancer may be selected from HPV-16 positive squamous cell carcinoma of the head and neck (HPV-16+ SCCHN), HPV-16 positive vulvar cancer and HPV16-positive vaginal cancer. In this case, the poxvirus (preferably an MVA) preferably encodes HPV-16 E6 and E7 polypeptides (more preferably non-oncogenic versions thereof, as disclosed above).

In addition to its HPV-positive nature, the targeted cancer is further preferably a recurrent and/or metastatic HPV-positive cancer (more preferably a recurrent and/or metastatic HPV-16 positive cancer, most preferably recurrent and/or metastatic HPV-16 positive SCCHN). As used herein, the term “cancer” encompasses all of primary or recurrent and/or metastatic cancers. “Primary cancer” is meant to be a cancer growing at the original anatomical site (organ or tissue) where tumor progression began and proceeded to yield a cancerous mass. “Recurrent cancer” is meant to be a cancer that has recurred (come back), usually after a period during which the cancer could not be detected. Cancer cells from a primary cancer may spread to other parts of the body and form new or “metastatic cancer” (also referred to as secondary cancer).

Indeed, recurrent and/or metastatic cancers are generally associated to a worse prognosis and lower response to treatment, and new combination treatments for these cancers are particularly needed. Metastases may affect various organs, including lymph nodes, lungs, bones and liver.

In their pooled interim analysis of phase Ib and phase II clinical trials, the inventors surprisingly showed that lymph node metastase(s) is significantly associated to better PFS (see FIG. 8). Therefore, in a preferred embodiment, said HPV-positive cancer is HPV-positive (preferably HPV16-positive) metastatic cancer with lymph node metastasis.

Moreover, they also found that lung and bone metastases are surprisingly not significantly associated to lower ORR or PFS (see FIG. 8). The inventors in fact showed that only one type of metastases is significantly associated with lower ORR and PFS: liver metastases, and in particular when the patient has multifocal liver metastases, i.e. liver metastases occurring at multiple sites (at least 2) in the liver, in particular in at least two distinct lobes. The presence of liver metastases has been suggested as associated with lower response to anti-PD-L1 treatment, mainly in lung cancers or in mixed cancer patients (Sridhar S., et al. Clin Lung Cancer 2019; e601-e608; Bilen M., et al. BMC Cancer. 2019; 19: 857; Reck M., et al. Lancet Respir Med 2019; 7: 387-401). However, such observations have been made with treatments that did not involve combination with a poxvirus vector encoding at least human papillomavirus (HPV) E6 and E7 polypeptides and an immunostimulatory cytokine, and transposition to the particular treatment according to the invention could thus not be expected. Moreover, such observations had not been made in HPV-positive cancers, and transposition to such specific cancers could also not be expected. In view of the inventors' observations concerning significant association of liver metastases to lower ORR and PFS, but absence of association of lung or bone metastases with lower ORR and PFS and association of lymph node metastases with higher PFS, the HPV-positive cancer may preferably be HPV-positive (preferably HPV-16 positive) cancer (such as oropharyngeal, cervical, vaginal, anal, vulvar, penile, mucosal, or non-melanoma skin cancer) without multifocal liver metastasis (preferably without liver metastasis), and in particular metastatic HPV-positive (preferably HPV-16 positive) cancer (such as oropharyngeal, cervical, vaginal, anal, vulvar, penile, mucosal, or non-melanoma skin cancer)_without multifocal liver metastasis (preferably without liver metastasis).

More preferably, the HPV-positive cancer may preferably be metastatic HPV-positive (preferably HPV-16 positive) cancer without liver metastasis and with lymph node metastasis.

### Doses and Administration Routes

“Administering” or “administration of” a drug to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a drug or provides a patient with a prescription for a drug is administering the drug to the patient.

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients, such as carriers, or adjuvants.

“Pharmaceutically acceptable adjuvant” refers to any and all substances which enhance the body's immune response to an antigen. Non-limiting examples of pharmaceutically acceptable adjuvants are: Alum, Freund's Incomplete Adjuvant, MF59, synthetic analogs of dsRNA such as poly(I:C), bacterial LPS, bacterial flagellin, imidazolquinolines, oligodeoxynucleotides containing specific CpG motifs, fragments of bacterial cell walls such as muramyl dipeptide and Quil-A®.

“Pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the pharmaceutical composition.

“Therapeutically effective amount” refers to an amount of the poxvirus described herein (preferably VV, more preferably MVA encoding at least HPV E6 and E7 polypeptides and an immunostimulatory cytokine such as TG4001 described in WO1999/03885 under its research name MVATG8042), and/or anti-PD-L1 antibody or antigen-binding fragment thereof (such as avelumab), which has a therapeutic effect and the capability of treating cancer or precancerous lesions. In the case of cancer, e.g., an advanced solid malignancy, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (UP) or any combination thereof. To the extent the drug can prevent growth and/or kill existing cancer cells, it can be cytostatic and/or cytotoxic. In the case of precancerous lesions, the therapeutically effective amount of the drug can inhibit (i.e., slow to some extent and in a certain embodiment, stop) evolution to cancer. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

“Unit dosage form” as used herein refers to a physically discrete unit of therapeutic formulation appropriate for the subject to be treated. It will be understood, however, that the usage of the poxvirus vector and anti-PD-L1 compositions described herein will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### Poxvirus

In the combination, method or use according to the invention, the poxvirus encoding at least human papillomavirus (HPV) E6 and E7 polypeptides and an immunostimulatory cytokine (preferably an MVA virus encoding membrane anchored non-oncogenic HPV-16 E6 and E7 polypeptides and human IL-2, as represented by TG4001 described in WO 1999/03885 under its research name MVATG8042) is preferably administered at a dose of 10⁶ to 10⁸ pfu, more preferably 5×10⁶ to 8×10⁷ pfu, most preferably 3×10⁷ to 7×10⁷ pfu, highly preferably 4×10⁷ to 6×10⁷ pfu, particularly highly preferably about 5×10⁷ pfu.

In the combination, method or use according to the invention, the poxvirus encoding at least human papillomavirus (HPV) E6 and E7 polypeptides and an immunostimulatory cytokine (preferably an MVA virus encoding membrane anchored non-oncogenic HPV-16 E6 and E7 polypeptides and human IL-2, as represented by TG4001 described in WO 1999/03885 under its research name MVATG8042) is preferably administered by subcutaneous, intramuscular, intratumoral or intravenous route. A particularly preferred administration route is the subcutaneous route.

### Anti-PD-L1 Antibody or Antigen-Binding Fragment Thereof

In the combination, method or use according to the invention, the anti-PD-L1 antibody (particularly, an antibody containing at least the 6 CDRs or the heavy and light chains of, e.g., avelumab) is preferably administered at a dose of:

In certain embodiments, a therapeutically effective amount of an anti-PD-L1 antibody (e.g., avelumab), or antigen-binding fragment thereof, is administered in the combinations, methods or use of the invention. The therapeutically effective amount is sufficient for treating one or more symptoms of an HPV-positive cancer. In some embodiments that employ an anti-PD-L1 antibody in the combination therapy, the dosing regimen will comprise administering the anti-PD-L1 antibody at a dose of about 1, 2, 3, 4, 5, 6, 7, 8, 9, 10, 11, 12, 13, 14, 15, 16, 17, 18, 19 or 20 mg/kg of body weight at intervals of about 7 days (±2 days), about 14 days (±2 days), about 21 days (±2 days) or about 30 days (±2 days) throughout the course of treatment. In certain embodiments, the therapeutically effective amount of anti-PD-L1 antibody (e.g., avelumab), or antigen-binding fragment thereof, is about 5 to 20 mg/kg, more preferably 5 to 15 mg/kg, most preferably 10 mg/kg. In some embodiments, the anti-PD-L1 antibody is avelumab and the therapeutically effective amount of avelumab is about 10 mg/kg. In some embodiments, the avelumab is administered once every two weeks. In some embodiments, the avelumab is administered on days 1 and 15 of a 28-day cycle. Pharmacokinetic studies demonstrated that the 10 mg/kg dose of avelumab achieves excellent receptor occupancy with a predictable pharmacokinetics profile (Heery et al., 2015. Proc ASCO Annual Meeting: abstract 3055). This dose is well tolerated, and signs of antitumor activity, including durable responses, have been observed.

In some embodiment, the anti-PD-L1 antibody (e.g., avelumab) is administered as a flat dose of about 80, 150, 160, 200, 240, 250, 300, 320, 350, 400, 450, 480, 500, 550, 560, 600, 640, 650, 700, 720, 750, 800, 850, 880, 900, 950, 960, 1000, 1040, 1050, 1100, 1120, 1150, 1200, 1250, 1280, 1300, 1350, 1360, 1400, 1440, 1500, 1520, 1550 or 1600 mg, preferably 800 mg, 1200 mg or 1600 mg at intervals of about 7 days (±2 days), about 14 days (±2 days), about 21 days (±2 days) or about 30 days (±2 days) throughout the course of treatment mentioned above and below. In a preferred embodiment, the anti-PD-L1 antibody (e.g., avelumab) is preferably administered once every week (QW), every two weeks (Q2W) or every three weeks (Q3W), at a dose of about 400 to 1600 mg, more preferably about 800 to 1600 mg, most preferably about 800 to 1200 mg, highly preferably about 800 mg. In a particular preferred embodiment, the anti-PD-L1 antibody (e.g., avelumab) is administered at a dose of about 800 mg Q2W.

In the combination, method or use according to the invention, the anti-PD-L1 antibody (particularly, an antibody containing at least the 6 CDRs or the heavy and light chains of, e.g., avelumab) is preferably administered intravenously (e.g., as an intravenous infusion) or subcutaneously. More preferably, the anti-PD-L1 antibody (particularly, an antibody containing at least the 6 CDRs or the heavy and light chains of, e.g., avelumab) is administered as an intravenous infusion. Most preferably, the anti-PD-L1 antibody (particularly, an antibody containing at least the 6 CDRs or the heavy and light chains thereof, e.g., avelumab) is administered for 50-80 minutes, highly preferably as an about one-hour intravenous infusion.

In one embodiment, avelumab is a sterile, clear, and colorless solution intended for IV administration. The contents of the avelumab vials are non-pyrogenic, and do not contain bacteriostatic preservatives. Avelumab is formulated as a 20 mg/mL solution and is supplied in single-use glass vials, stoppered with a rubber septum and sealed with an aluminum polypropylene flip-off seal. For administration purposes, avelumab must be diluted with 0.9% sodium chloride (normal saline solution). Tubing with in-line, low protein binding 0.2 micron filter made of polyether sulfone (PES) is used during administration.

### Number and Frequency of Combination Administrations

In an aspect of the invention, the combination of:

a) a poxvirus vector encoding at least human papillomavirus (HPV) E6 and E7 polypeptides and an immunostimulatory cytokine, preferably an MVA virus encoding membrane anchored non-oncogenic HPV-16 E6 and E7 polypeptides and human IL-2, more preferably TG4001, and

b) an anti-PD-L1 antibody or antigen-binding fragment thereof, preferably avelumab,

is administered according to specific administration schemes, in which a first administration of said poxvirus is performed 5 to 10 days before the first administration of said anti-PD-L1 antibody, and subsequent administrations of said poxvirus and anti-PD-L1 antibody are performed.

In other words, the administration scheme used for the combination, method or use according to the invention involves at least:
a first administration of said poxvirus,
which is performed 5 to 10 days before the first administration of said anti-PD-L1 antibody, and
subsequent administrations of said poxvirus and anti-PD-L1 antibody.

In the administration scheme used for the combination, method or use according to the invention, a first administration of said poxvirus is performed before the first administration of said anti-PD-L1 antibody. Without being bound by theory, this setting first stimulates an anti-HPV immune response by the first poxvirus administration and then amplifies this anti-HPV immune response by the first anti-PD-L1 administration (by reducing immune suppression due to the PD-1/PD-L1 pathway in the tumor microenvironment), without altering poxvirus initial propagation. Absence of anti-PD-L1 administration in the 5 to 10 days after first poxvirus administration prevents potential amplification of anti-poxvirus immune response. Subsequent administrations of said poxvirus and anti-PD-L1 antibody sustain the anti-HPV immune response.

Thus, in the combination, method or use according to the invention, the first administration of the poxvirus is performed about 5 to 10 days (i.e., 5, 6, 7, 8, 9 or 10 days, preferably 1 week) before the first administration of said anti-PD-L1 antibody.

In some embodiments, the combination regimen comprises the steps of: (a) under the direction or control of a physician, the subject receiving the poxvirus vector encoding at least HPV E6 and E7 polypeptides and an immunostimulatory cytokine about 5 to 10 days prior to first receipt of the PD-L1 antibody; and (b) under the direction or control of a physician, the subject receiving the PD-L1 antibody. In some embodiments, the combination regimen comprises, administering the anti-PD-L1 antibody to the subject about 5 to 10 days after the subject has received the first administration of the poxvirus vector encoding at least HPV E6 and E7 polypeptides and an immunostimulatory cytokine.

The number and frequency of the subsequent administrations of said poxvirus and anti-PD-L1 antibody may vary. However, with respect to their number, subsequent administrations of said poxvirus and anti-PD-L1 antibody are preferably performed as long as the combination treatment results in beneficial effects in the treated subject without inducing unacceptable toxicities.

In an embodiment, subsequent administrations of said poxvirus and anti-PD-L1 antibody may be performed until disease progression, which is defined according to RECIST v1.1 criteria (Eisenhauer E A. et al., Eur J Cancer. (2009) 45(2):228-47).

“Disease progression”, “progressive disease” or “disease that has progressed” refers to the appearance of one more new lesions or tumors and/or the unequivocal progression of existing non-target lesions as defined in the RECIST v1.1 guideline. Disease progression, progressive disease or disease that has progressed can also refer to a tumor growth of more than 20 percent since treatment began, either due to an increase in mass or in spread of the tumor.

“RECIST” means Response Evaluation Criteria in Solid Tumors. RECIST guideline, criteria, or standard, describes a standard approach to solid tumor measurement and definitions for objective assessment of change in tumor size for use in adult and pediatric cancer clinical trials. RECIST v1.1 means version 1.1 of the revised RECIST guideline.

In other embodiments, subsequent administrations of said poxvirus and anti-PD-L1 antibody may be performed as long as beneficial biological effects (see dedicated section below) are observed in the patient.

With respect to the frequency of administrations, the following schedules are preferably used:

The frequency of subsequent poxvirus administrations may vary between about 1 week to about 3 months. Moreover, the frequency of poxvirus administrations may not be constant during the whole duration of treatment but may instead vary. Preferably, the frequency of subsequent poxvirus administrations is reduced over time.

In particular, when including the first poxvirus administration, 4 to 8 (i.e., 4, 5, 6, 7 or 8, preferably 6) poxvirus administrations every 5 to 10 days (including every 5, 6, 7, 8, 9 or 10 days, preferably on a weekly basis) may be administered first (optionally, with a specific preference for a single first poxvirus administration followed by 5 subsequent poxvirus administrations on a weekly basis (the “first group of poxvirus administrations”).

This first group of poxvirus administrations may then be followed by a second group of subsequent poxvirus administrations with reduced frequency. Preferably, this second group of subsequent poxvirus administrations comprises 6 to 10 (i.e., 6, 7, 8, 9 or 10, preferably 8) subsequent poxvirus administrations every 1 to 3 weeks (including every 1, 2 or 3 weeks, preferably every 2 weeks) (optionally, with a specific preference for subsequent poxvirus administrations every 2 weeks up to month 6) (the “second group of poxvirus administrations”).

This second group of poxvirus administrations may then be followed by a third group of subsequent poxvirus administrations with further reduced frequency. Preferably, this third group of subsequent poxvirus administrations may be administered every 10-14 weeks (including every 10, 11, 12, 13 or 14 weeks, preferably every 12 weeks) until disease progression (or optionally, as long as at least one of the biological effects described herein below is present) (the “third group of poxvirus administrations”).

In a particularly preferred embodiment, poxvirus is administered:
on a weekly basis for 6 weeks,
every 2 weeks up to month 6, and
every 12 weeks, until disease progression (or optionally, as long as at least one of the biological effects described herein below is present).

The frequency of anti-PD-L1 administrations (including the first administration and the subsequent administrations) is preferably between 1 and 3 weeks (including every week, or every 2 or 3 weeks, preferably every 2 weeks).

The anti-PD-L1 antibody will preferably be administered until disease progression (or optionally, as long as at least one of the biological effects described herein below is present).

In a particularly preferred embodiment, the anti-PD-L1 antibody is administered every 2 weeks until disease progression (or optionally, as long as at least one of the biological effects described herein below is present).

### Preferred Administration Schemes

In a preferred embodiment, the combination is administered with the following administration scheme:
a) a first dose of 3×10⁷ to 7×10⁷ pfu of said poxvirus (preferably, an MVA virus encoding membrane anchored non-oncogenic HPV-16 E6 and E7 polypeptides and human IL-2, more preferably TG4001, as described, e.g., in WO 1999/03885 under its research name MVATG8042) is administered subcutaneously, and followed until disease progression by subsequent poxviruses doses of 3×10⁷ to 7×10⁷ pfu administered subcutaneously:
   on a weekly basis for 6 weeks,
   once every 2 weeks up to month 6, and
   every 12 weeks, for the next poxviruses doses;
b) a first dose of about 10 mg/kg or about 800 mg of anti-PD-L1 antibody (preferably, an antibody containing at least the 6 CDRs or the heavy and light chains of, e.g. avelumab, more preferably avelumab) is administered intravenously 5 to 10 days after the first poxvirus dose, and followed by subsequent anti-PD-L1 antibody doses of about 10 mg/kg or about 800 mg, which are administered intravenously every 2 weeks until disease progression.

In an even more preferred embodiment, the combination is administered with the following administration scheme:
a) a first dose of about 5×10⁷ pfu of said an MVA virus encoding membrane anchored non-oncogenic HPV-16 E6 and E7 polypeptides and human IL-2 (preferably TG4001, as described, e.g., in WO 1999/03885 under its research name MVATG8042) is administered subcutaneously, and followed until disease progression by subsequent MVA doses of about 5×10⁷ pfu administered subcutaneously:
   on a weekly basis for 6 weeks,
   once every 2 weeks up to month 6, and
   every 12 weeks, for the next poxviruses doses;
b) a first dose of about 10 mg/kg or about 800 mg of avelumab is administered intravenously 1 week after the first poxvirus dose, followed by subsequent avelumab doses of about 10 mg/kg or about 800 mg, which are administered intravenously every 2 weeks until disease progression.

### Biological Effects of the Combination Treatment and Biomarkers

The inventors surprisingly found that combining (a) a poxvirus vector encoding at least human papillomavirus (HPV) E6 and E7 polypeptides and an immunostimulatory cytokine, particularly TG4001 (an MVA virus encoding membrane anchored non-oncogenic HPV-16 E6 and E7 polypeptides and human IL-2) and (b) an anti-PD-L1 antibody or antigen-binding fragment thereof, particularly avelumab, in HPV-positive cancer patients results in reduced tumor immune suppression and improved anticancer responses, characterized by:
an induction of or an increase in an immune response against HPV16 E6 and E7 polypeptides;
within the tumor:
   an increase in immune cell infiltrates, mainly of CD3 T cells, preferably an increase in the number of CD8 T cells and in their proportion among CD3 T cells (increase in the CD8/CD3 ratio) and/or a decrease in regulatory CD4 T cells, and/or the combined increase in CD8 T cells and decrease in regulatory CD4 T cells (Treg) resulting in a decrease in the Treg/CD8 ratio; and/or
   an increase of PD-L1 expression on tumor cells;
in the blood circulation:
   an increase in CD8 T cells; and/or
   a decrease in regulatory CD4 T cells; and/or
a significant remodeling of gene expression in tumor cells, characterized by:
   an increase in the expression of T cell activation genes, cytotoxic cell genes, pathogen defense genes and NK cell function genes;
   an increase in the expression of one or more genes selected from the group of CXCL10, CXCL11, IRF1, GZMK, GZMA, CD3D, PRF1, TBX21, CXCR3, STAT1, CD69, CCL2, GZMB, CD3G, ICOS, CD8A, STAT4, GZMM, CCR2, CD3E and IL15; and/or
   an increase in the expression of one or more genes selected from the group of CXCL13, GNLY, GZMH, IFNG, CXCL9, CCLS and ITGAE, and/or a decrease in the expression of one or more of VEGFA, IHH, IL17A, PROM1, REN, PF4, TSLP and LAG3.

Except otherwise stated, all comparisons for an increase or decrease are made compared to baseline (i.e., before the combination therapy is administered to the patient).

Therefore, in an embodiment of the combination therapy, said combination induces an induction of or an increase in an immune response against HPV-16 E6 and E7 proteins. The immune response against HPV-16 E6 and E7 proteins may be measured by any suitable method known in the art. Suitable methods may be based on detection of CD8 and/or CD4 T cells responses against HPV-16 E6 and E7 proteins, including cytokine (notably interferon gamma (IFNγ), interleukin-2 (IL-2), and tumor necrosis factor alpha (TNFα)) secretion and cytotoxicity. Cytokine secretion may be measured in vitro from a peripheral blood mononuclear cell (PBMC) sample using conventional assays such as ELISA or ELISPOT. Cytotoxicity may also be measured in vitro using conventional assays. In a preferred embodiment, the measured immune against HPV-16 E6 and E7 proteins will be the secretion of IFNγ by PBMC, and will be measured using ELISA, immunostaining by flow cytometry or ELISPOT, preferably ELISPOT technique.

In another embodiment of the combination therapy, said combination induces within the tumor:
an increase in immune cell infiltrates, preferably an increase in CD3 T cells, more preferably an increase in CD8 T cells, and most preferably an increase in the CD8/CD3 ratio; and/or
a decrease in regulatory CD4 T cells (Treg), more preferably a decrease in the Treg/CD3 ratio.

Most preferably, said combination induces a decrease in the Treg/CD8 ratio within the tumor. Such decrease in the Treg/CD8 ratio is indicative of reduced immune suppression within the tumor and stimulation of anticancer immune response.

Within the tumor, immune cell infiltrates, and in particular the number of CD3 T cells, CD8 T cells and CD4 T cells (Treg) may be characterized by any suitable method known in the art. T cells are characterized by the surface expression of CD3, and are subdivided in two subgroups depending on their concomitant surface expression of either CD8 or CD4. Among CD4 T cells, those further expressing Foxp3 are considered regulatory

CD4 T cells (Treg). The numbers of CD3 T cells (CD3+ cells), CD8 T cells (CD3+CD8+ cells), and CD4 T cells (Treg, CD3+CD4+Foxp3+ cells) may be measured using any suitable method known in the art before (at baseline) and after treatment and compared. The CD8/CD3 ratio, Treg/CD3 ratio and Treg/CD8 ratio may then be easily calculated.

As used herein, CD3, CD8, CD4 and Foxp3 expression means any detectable level of expression of CD3, CD8, CD4 and Foxp3 protein on the cell surface or of CD3, CD8, CD4 and Foxp3 mRNA within a cell or tissue. CD3, CD8, CD4 and/or Foxp3 protein expression on the cell surface may be detected with diagnostic CD3, CD8, CD4 and/or Foxp3 antibodies in an immunohistochemistry (IHC) assay of a tumor tissue section or by flow cytometry, depending on the type of sample. Alternatively, CD3, CD8, CD4 and/or Foxp3 protein expression by tumor cells may be detected by PET imaging, using a binding agent (e.g., antibody fragment, affibody and the like) that specifically binds to CD3, CD8, CD4 and/or Foxp3. Techniques for detecting and measuring CD3, CD8, CD4 and/or Foxp3 mRNA (or cDNA) expression include RT-PCR, real-time quantitative RT-PCR (qRT-PCR) and microarray hybridization. Within the tumor, CD3, CD8, CD4 and/or Foxp3 expression will preferably be detected with diagnostic CD3, CD8, CD4 and/or Foxp3 antibodies in an immunohistochemistry (IHC) assay of a tumor tissue section. An increase/decrease is detected when number, expression or ratio after treatment with the combination therapy is higher/lower than before (at baseline) treatment with the combination therapy.

In another embodiment of the combination therapy, said combination induces an increase of PD-L1 expression on tumor cells. “PD-L1 expression” as used herein means any detectable level of expression of PD-L1 protein on the cell surface or of PD-L1 mRNA within a cell or tissue. PD-L1 protein expression may be detected with a diagnostic PD-L1 antibody in an immunohistochemistry (IHC) assay of a tumor tissue section or by flow cytometry, depending on the type of sample. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA (or cDNA) expression include RT-PCR, real-time quantitative RT-PCR (qRT-PCR) and microarray hybridization. Within the tumor, PD-L1 expression will preferably be detected with a diagnostic PD-L1 antibody in an immunohistochemistry (IHC) assay of a tumor tissue section. An increase is detected when PD-L1 expression after treatment with the combination therapy is higher than before (at baseline) treatment with the combination therapy. A high level of PD-L1 expression on tumor cells has been associated to better clinical response to anti-PD-L1 antibody treatment.

In another embodiment of the combination therapy, said combination induces in the blood circulation:
an increase in CD8 T cells, preferably an increase in the CD8/CD3 ratio; and/or
a decrease in regulatory CD4 T cells (Treg), more preferably a decrease in the Treg/CD3 ratio.

In the blood circulation, CD3, CD8, CD4 and Foxp3 expression may be measured using any suitable method known in the art before (at baseline) and after treatment, and the expression levels are compared. Suitable methods include the same as those disclosed above for measuring CD3, CD8, CD4 and Foxp3 expression within the tumor, but with a preference for the detection of CD3, CD8, CD4 and Foxp3 expression using diagnostic CD3, CD8, CD4 and/or Foxp3 antibodies by flow cytometry. The CD8/CD3 ratio, Treg/CD3 ratio and Treg/CD8 ratio may then be easily calculated.

Preferably, an increase in CD8 T cells is observed in both the blood circulation and within the tumor. Similarly, a decrease in regulatory CD4 T cells is preferably observed in both the blood circulation and within the tumor. Moreover, both an increase in CD8 T cells and a decrease in regulatory CD4 T cells is preferably observed, in the blood circulation and/or within the tumor, more preferably both in the blood circulation and within the tumor.

Moreover, at the molecular level, the inventors also surprisingly found gene expression changes that are consistent with a priming of innate and adaptive immunity and shift from a “cold” tumor profile to a “hot” tumor profile. A “cold tumor” is defined as a tumor lacking or with very limited immune infiltrates, in particular T cells infiltrates. At the molecular level, a “cold tumor” is characterized by a low level of expression of genes associated to the presence of immune cells infiltrates, and in particular of genes related to T-cell activation, T-cell differentiation, T-cell attraction, T-cell adhesion, cytotoxicity, pathogen defense and NK cell function. In contrast, a “hot tumor” is defined as a tumor with significant immune infiltrates, in particular T cells infiltrates. At the molecular level, a “hot tumor” is characterized by a high level of expression of genes associated to the presence of immune cells infiltrates, and in particular of genes related to T-cell activation, T-cell differentiation, T-cell attraction, T-cell adhesion, cytotoxicity, pathogen defense and NK cell function. A shift from a “cold tumor” profile to a “hot tumor” profile is considered as induced by a treatment when the treatment results in a significant increase in immune infiltrates, in particular T cells infiltrates. At the molecular level, this is reflected by an increase in the level of expression of one or more genes related to T-cell activation, T-cell differentiation, T-cell attraction, T-cell adhesion, cytotoxicity, pathogen defense and/or NK cell function is increased compared to before treatment. Hot tumors are more likely to respond to therapeutic intervention and are more consistently associated with an improved clinical outcome for the patient. On the contrary, a cold tumor is associated with little immunoreactivity, poor response to therapeutic intervention and is likely to have a rapid unfavorable clinical course.

More particularly, using a panel of 770 genes related to immune response to cancer, the inventors were able to show variations in gene expression in the tumor between baseline and day 43 after start of the combination treatment. Such variations include an increase in the expression of several T cell activation genes, cytotoxic cell genes, pathogen defense genes and NK cell function genes. The variations also include an increase in the gene signatures known as Immunosign®_{CR} 15 and Immunosign®_{CR} 21 by HalioDX, which are considered to reflect the naturally occurring immune activity in and around the tumor, and thus the rather cold (bad prognosis) or hot (better prognosis) immune state of a tumor (Galon J. et al., Immunity (2013) 39(1):11-26; Marabelle A. et al., Society for Immunotherapy of Cancer (SITC) 32^{nd} Annual Meeting [symbol] Pre-Conference Programs (SITC 2017) on Nov. 8-12, 2017 at the Gaylord National Hotel [symbol] Convention Center in National Harbor, Maryland. Poster P250).

In detail, Immunosign®_{CR} 15 is an algorithm combining expression data of genes related to T-cell cytotoxicity, T-cell differentiation, T-cell attraction, T-cell adhesion, immune orientation, angiogenesis suppression, immune co-inhibition, and cancer stem cells: CXCL13, GNLY, GZMH, IFNG, CXCL9, CCL5, ITGAE, VEGFA, IHH, IL17A, PROM1, REN, PF4, TSLP, LAG3. In the context of the invention, an increase in the expression level of any one of CXCL13, GNLY, GZMH, IFNG, CXCL9, CCLS and ITGAE, and/or a decrease in the expression level of any one of VEGFA, IHH, IL17A, PROM1, REN, PF4, TSLP and LAG3 is considered as a shift to a hotter tumor status, beneficial for cancer treatment.

Immunosign®_{CR} 21 is an algorithm combining expression data of genes related to T-cell cytotoxicity, T-cell activation, T-cell attraction, and Th1 orientation: CXCL10, CXCL11, IRF1, GZMK, GZMA, CD3D, PRF1, TBX21, CXCR3, STAT1, CD69, CCL2, GZMB, CD3G, ICOS, CD8A, STAT4, GZMM, CCR2, CD3E and IL15. In the context of the invention, an increase in the expression level of any one of these genes is considered as a shift to a hotter tumor status, beneficial for cancer treatment.

Therefore, in one aspect of the invention, the combination therapy induces an increased expression in one or more of the following gene categories (surprisingly found by the inventors to be upregulated by the combination therapy):
(i) an increase in expression of at least one gene related to T cell activation, preferably selected from the following group of CD47, RPS6, CD80, IL18R1, CD7, PSEN2, TNFSF14, DPP4, STAT4, CCR1, FOXP3, CTLA4, LAG3, CD86, LILRB1, IL13, CD1C, EOMES, CCR4, CD3G, FAS, IL12B, IL18RAP, CD1D, CXCR3, TIGIT, IL4, IL12A, IFNG, CD70, CD2, CD3E, CD8A, CD8B, IL12RB2, CDS, CCR5, TBX21, IL12RB1, IRF4, ADA, CD274, LCK, F2RL1, ICOSLG, CXCL11, CXCL10, IDO1, CX3CL1, IRF1, SOCS1, IL18, SLC11A1, EGR1, ITGA1, CXCR4, CXCL9, PTPRC, LCP1, TNFRSF14, PSEN1, MAF, TP53, IL4R, STAT6, IL13RA1 and IFNGR1, and optionally IL21R genes;
(ii) an increase in expression of at least one gene related to the activation of cytotoxic T cell function, preferably selected from the group of GZMM, GZMH, GZMK, GNLY, GZMB, PRF1, GZMA, HLA-C and HLA-A genes;
(iii) an increase in expression of at least one pathogen defense gene, preferably selected from the group of CD8A, CTSG, PRG2, CCL22, IL1B, PRF1, GNLY, CXCL10, TYK2 and OAS3 genes;
(iv) an increase in expression of at least one NK cell function gene, preferably selected from the group of KLRC1, KLRB1, KLRC2, IL12B, KIR3DL1, KLRF1, KLRG1, NCR1, KLRK1, IL12A and KLRD1 genes;
(v) an increase in expression of any gene combination of the gene categories (i), (ii), (iii) and/or (iv), preferably a gene combination comprising at least one gene of each of the above-mentioned gene categories.

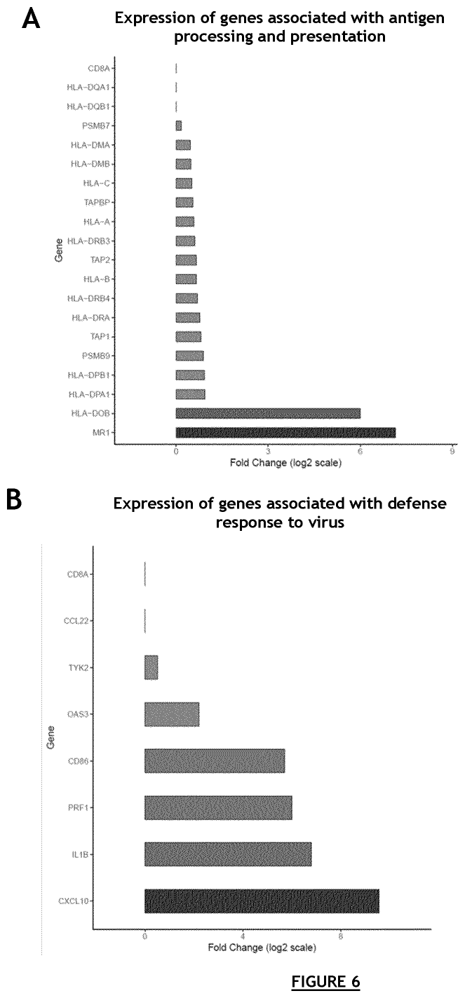

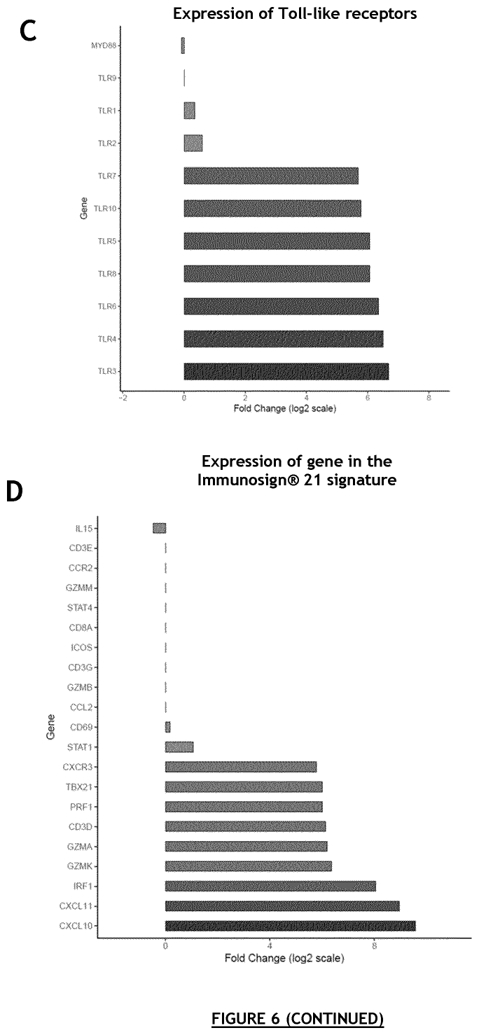

In another aspect of the invention, alternatively or in addition to the gene expressions of categories (i) to (v) above, the combination therapy induces an increased expression in one or more of the following genes(present in the Immunosign® 21 signature): CXCL10, CXCL11, IRF1, GZMK, GZMA, CD3D, PRF1, TBX21, CXCR3, STAT1, CD69, CCL2, GZMB, CD3G, ICOS, CD8A, STAT4, GZMM, CCR2, CD3E and IL15. In particular, the combination therapy may induce an increased expression in one or more of the following genes (see FIG. 6D): CXCL10, CXCL11, IRF1, GZMK, GZMA, CD3D, PRF1, TBX21 and CXCR3.

In another aspect of the invention, alternatively or in addition to the gene expressions of categories (i) to (v) and/or the genes of the Immunosign® 21 signature above, the combination therapy induces an increased or decreased expression in one or more of the following genes (present in the Immunosign® 15 signature), as follows:
an increased expression in one or more of the following genes: CXCL13, GNLY, GZMH, IFNG, CXCL9, CCLS and ITGAE, and/or
a decreased expression in one or more of the following genes: VEGFA, IHH, IL17A, PROM1, REN, PF4, TSLP and LAG3.

In the above embodiments, the expression level of the disclosed gene categories or specific genes of interest may be measured using any suitable method known in the art before (at baseline) and after treatment, and the expression levels are compared. The expression level may be measured by measuring either the mRNA (or cDNA) or protein expression level. Preferably, the mRNA (or cDNA) expression level is measured by techniques such as RT-PCR, real-time quantitative RT-PCR (qRT-PCR) and microarray hybridization.

The above-described biological effects of the combination therapy may also be used as biomarkers before or during the combination therapy.

In an embodiment, they may notably be used as biomarkers during the combination therapy, to decide whether to continue or stop the combination therapy in a patient.

In an embodiment where the biomarkers are used to decide whether to continue or stop the combination therapy in a patient, subsequent administrations of said poxvirus and anti-PD-L1 antibody may be performed as long as the combination treatment induces or increases an immune response against HPV-16 E6 and E7 proteins.

In another embodiment, subsequent administrations of said poxvirus and anti-PD-L1 antibody may be performed as long as the combination treatment induces:
an increase in immune cell infiltrates (with a preference for an increase in CD3 T cells, more preferably an increase in CD8 T cells) within the tumor; and/or
a decrease in regulatory CD4 T cells within the tumor.

In another embodiment, subsequent administrations of said poxvirus and anti-PD-L1 antibody may be performed as long as the combination treatment induces an increase of PD-L1 expression on tumor cells.

In another embodiment, subsequent administrations of said poxvirus and anti-PD-L1 antibody may be performed as long as the combination treatment induces in the blood circulation:
an increase in CD8 T cells, preferably an increase in the CD8/CD3 ratio; and/or
a decrease in regulatory CD4 T cells (Treg), more preferably a decrease in the Treg/CD3 ratio.

In another embodiment, subsequent administrations of said poxvirus and anti-PD-L1 antibody may be performed as long as the combination treatment induces an increased expression in one or more of the following gene categories (surprisingly found by the inventors to be upregulated by the combination therapy):
(i) an increase in expression of at least one gene related to T cell activation, preferably selected from those disclosed above;
(ii) an increase in expression of at least one gene related to the activation of cytotoxic T cell function, preferably selected from those disclosed above;
(iii) an increase in expression of at least one pathogen defense gene, preferably selected from those disclosed above;
(iv) an increase in expression of at least one NK cell function gene, preferably selected from those disclosed above;
(v) an increase in expression of any gene combination of the gene categories (i), (ii), (iii) and/or (iv), preferably a gene combination comprising at least one gene of each of the above-mentioned gene categories.

In another embodiment, alternatively or in addition to the gene expressions of categories (i) to (v) above, subsequent administrations of said poxvirus and anti-PD-L1 antibody may be performed as long as the combination treatment induces an increased expression in one or more of the following genes (present in the Immunosign® 21 signature): CXCL10, CXCL11, IRF1, GZMK, GZMA, CD3D, PRF1, TBX21, CXCR3, STAT1, CD69, CCL2, GZMB, CD3G, ICOS, CD8A, STAT4, GZMM, CCR2, CD3E and IL15. In particular, subsequent administrations of said poxvirus and anti-PD-L1 antibody may be performed as long as the combination treatment induces an increased expression in one or more of the following genes (see FIG. 6D): CXCL10, CXCL11, IRF1, GZMK, GZMA, CD3D, PRF1, TBX21 and CXCR3.

In another embodiment, alternatively or in addition to the gene expressions of categories (i) to (v) and/or the genes of the Immunosign® 21 signature above, subsequent administrations of said poxvirus and anti-PD-L1 antibody may be performed as long as the combination treatment induces an increased or decreased expression in one or more of the following genes (present in the Immunosign® 15 signature), as follows:
an increased expression in one or more of the following genes: CXCL13, GNLY, GZMH, IFNG, CXCL9, CCLS and ITGAE, and/or
a decreased expression in one or more of the following genes: VEGFA, IHH, IL17A, PROM1, REN, PF4, TSLP and LAG3.

All the references cited herein are incorporated by reference in the disclosure of the invention hereby.

The following examples merely intend to illustrate the present invention.

### EXAMPLES

### Example 1: Results of Phase Ib Clinical Trial NCT03260023

In NCT03260023, the combination of TG4001 and avelumab in HPV16-positive R/M (R/M for recurrent and/or metastatic) cancers was assessed in terms of safety, efficacy and immunological response. In this Example, preliminary data of the Phase Ib is presented.

### Materials and Methods

### Study Design and Treatments

Multicenter, open label, single arm study with a 3+3 design for the phase Ib, combining two different dose levels (DL) of TG4001 (DL1 5×10⁶ and DL2 5×10⁷ pfu) with avelumab at 10 mg/kg. For the phase II, TG4001 was administered at DL2.

TG4001 was administered subcutaneously (SC) on a weekly basis on Days 1, 8, 15, 22, 29 and 36, then once every 2 weeks (starting on Day 36) until Month 6 (from Day 1 of study treatment), thereafter once every 12 weeks, until disease progression, unacceptable toxicity, or patient withdrawal from study for any reason, whichever occurs first. Avelumab was given intravenously (IV infusion) every 2 weeks starting from Day 8 (one week after the first TG4001 dose), until disease progression, unacceptable toxicity, or patient withdrawal from study for any reason, whichever occurs first.

### Study Endpoints and Assessments

Safety and efficacy of the combination of TG4001 and avelumab, immune parameters (T cell response, changes in infiltrates and gene expression of immune related genes).

Tumor response was assessed by RECIST v1.1 (Eisenhauer E A. et al., Eur J Cancer (2009) 45(2):228-47). PBMC samples were collected longitudinally and tissue samples were collected at baseline and day 43.

### Study Population

Key Inclusion Criteria:
Metastatic or refractory/recurrent (M/R) HPV16+ cancer including oropharyngeal SCCHN, cervical, vulvar, vaginal, penile and anal cancer
HPV16 positivity determined in a central laboratory by detecting HPV-16 E7 DNA by PCR using HPV-16 specific primers
Up to two prior lines of systemic therapy for the management of metastatic or recurrent disease
ECOG Performance status 0 or 1

Key Exclusion Criteria:
Prior exposure to cancer immunotherapy including anti-cancer vaccines, any antibody targeting T cell co-regulatory proteins such as anti-PD L1, anti-PD 1, or anti-CTLA-4 antibodies
CNS metastases
Chronic treatment with systemic corticosteroids

### Tumor Size Measurement

Tumor response was evaluated by Computed Tomography (CT) (or Magnetic Resonance Imaging (MRI)). Preferably, CT (or MRI) of the head and neck, chest, abdomen and all other known sites of disease were performed within 21 days prior to start of study treatment. Patients were evaluated every 6 weeks from start of treatment until disease progression or for a period of 9 months after start of study treatment, whichever occurred first. Beyond 9 months of treatment, the evaluations were performed every 12 weeks until documented progression. All measurements were recorded in metric notation (mm).

At baseline, tumor lesions and lymph nodes were categorized as measurable (minimum size not less than 10 mm or lymph node >15 mm) or non-measurable (small lesions <10 mm, non-measurable lesions (e.g. pleural effusion) or lymph node <15 mm). Patients allowed to enter the study had at least one measurable lesion by CT/MRI scan. All target lesions (all measurable lesions (nodal or non-nodal) up to a maximum of lesions in total) and non-target lesions (all other lesions measurable or not) were recorded. To assess tumor response, the sum of the longest diameters (SLD) for all target lesions (and short axis for nodal lesions) were calculated at baseline and throughout the study. At each assessment, response was evaluated first separately for the target lesions and non-target lesions identified at baseline. These evaluations were then used to calculate the overall lesion response considering the target and non-target lesions as well as the presence or absence of new lesions:
Complete Response (CR): Disappearance of all target lesions. Any pathological lymph nodes (whether target or non-target) must have reduction in short axis to <10 mm.
Partial Response (PR): Decrease of at least 30% in the sum of the diameters of target lesions, taking as reference the baseline sum of diameters.
Progressive Disease (PD): Increase of at least 20% in the sum of diameters of target lesions and new lesions, taking as reference the smallest sum on study (including baseline evaluation). In addition to the relative increase of 20%, the sum must also demonstrate an absolute increase of at least 5 mm. The appearance of one or more new lesions is also considered as progression.
Stable Disease (SD): Neither sufficient shrinkage to qualify for PR nor sufficient increase to qualify for PD.
Not Evaluated (NE): Progression has not been documented and one or more target lesions have not been assessed or have been assessed using a different method than baseline. The only exception is if the SLD of the evaluable target lesions already qualifies for PD. In this case, the Target Response will be PD.

Evaluable patients for tumor response were all included patients who had at least one baseline and one post-baseline evaluable CT-scan at week 6 after start of study treatment with a best overall response assessment different from ‘Unknown’ according to RECIST 1.1 evaluation criteria. Patients were to be dosed with both IMPs (Investigational Medicinal Products: TG4001+avelumab) with a minimum exposure to be met except if patient had progressed or died due to underlying disease before or at the first evaluation.

### Immune Data

Samples were collected from consenting patients, in compliance with all ethical guidelines pertaining to human subject research and under after IRB approval. Peripheral Blood Mononuclear Cells (PBMC) were isolated using by density gradient on a Ficoll® layer. Briefly, heparinized blood was mixed with Phosphate-buffer saline and Ficoll® medium. Then, it was centrifuged at 2300 g for 20 minutes. The PBMC layer was collected, diluted with buffered saline and centrifuged 10 min at 1300 g to remove the remaining Ficoll® solution. Cell were resuspended in buffered saline and recentrifuged. The cell pellet was then resuspended in storage media (IMDM with 10% DMSO and 20% human serum), distributed in cryovials and frozen in a container using isopropyl alcohol.

Tissue samples were obtained using standard core needle biopsy using a needle of 18 G or above. Sample section of 4 μm (immunohistochemistry) or 10 μm (gene expression analysis) thickness were formalin-fixed paraffin embedded prior to processing.

### ELISPOT T Cell Responses Against HPV

In patients from the phase I study, IFN-γ producing cells were quantified by ELISpot after an in vitro expansion phase of 5 days. Briefly, after thawing, cells were counted with an NC200 automated cell counter and seeded at 2E+06 cells per 500 μL per well of 24-well culture plates in X-VIVO-15 medium containing 2% CTS Serum replacement solution and in the presence or not of stimulating antigens (E6 or E7 peptide pools at 2 μg/mL per peptide or a control peptide mix pool at 1 μg/mL). At day 5, cells were harvested, counted, and plated at 2E+05 cells per well of ELISpot IFN-γ plates in quadruplicates. After 24 H incubation, ELISpot plates were revealed according to the manufacturer's instructions, then dried before spot counting with an automated ELISPOT reader.

In patients from the phase II study, the method was modified to increase specificity. IFN-γ producing cells were quantified by ELISpot. Briefly, PBMC were collected by venipuncture in patients at Day 0, day 1 (pre-vaccination) and D43 (post-vaccination) into PCT tubes and shipped to a central lab (PPD) for extraction by centrifugation on a ficoll gradient. Cells were washed, counted and dispatched in tubes containing 10×10⁶ cells. Cells were frozen and stored in LN prior to analysis.

Cells were unfrozen and stimulated overnight with media (negative control), E6 peptide pool (PepTivator, Miltenyi Biotech), E7 peptide pool (PepTivator, Miltenyi Biotech) or CEF (PepTivator, Miltenyi Biotech; positive control: EBV, CMV, Flu). Cells were then plated on anti-IFN-γ coated plates and incubated (1×10⁵ cell/well) before revelation. PBMC collected in a patient prior and after peptide vaccination and known to have developed a T-cell response were used in each analytical run as an assay control. Three replicates were performed for each condition. After overnight incubation, ELISpot plates were revealed according to the manufacturer's instructions, then dried before spot counting with an automated ELISPOT reader. Patients were considered positive for an antigen when positive and negative controls were as expected, and when antigen spot counts were higher than negative control plus 2-fold the coefficient of variation of the assay.

### Immunohistochemistry for Immunoscore Assessment

Formalin fixed paraphing embedded tissue sections of 4 μm of thickness were used for characterization of the tumor immune contexture. Consecutive slices from the same biopsy core were used for all analysis on a given patient at baseline and at day 43. The first slide of each series was used for confirmation of the tumoral nature by pathologist review of the tissue after haematoxylin and eosin staining. CD3 and CD8 stainings were performed on consecutive slides according to the following protocol: antigen retrieval with a Tris base buffer (pH=8) for 60 min, quenching of endogenous peroxidase activity, incubation with antibody against CD8 for 32 min at 37° C. and with antibody against CD3 for 20 min at 37° C.; revelation with the Ultraview Universal DAB IHC Detection Kit, and counterstaining with Mayer's haematoxylin. Primary antibodies used for staining immune cells were the following: rabbit monoclonal anti-human CD3 VMS (clone 2GV6 Ventana), mouse monoclonal anti-human CD8 (clone C8/144, Dako®). Digital images of the stained tissue sections were obtained at 20× magnification and 0·45 μm/pixel resolution. Quantification of CD8 and CD3 positive cells in the tumor and in the invasive margin was performed from whole slide digital scans using the Immunoscore® module developed by HalioDx (Marseille, France).

### Multispectral Histologic Assessment for CD3/CD8/PD-L1 and CD3/CD4/FOXP3

4-mm-thick FFPE tissue slides were deparaffinized, rehydrated through an ethanol gradient ending with a distilled water wash and fixed in 10% neutral buffered formalin for 20 minutes. Antigen retrieval was performed via microwave treatment in antigen retrieval solution. At each of the cycles of staining, protein blocking was performed using Protein Block Serum-free solution for 15 min, and primary Abs anti-CD3 (obtained from Ventana as mentioned above), anti-CD4 (mouse monoclonal anti-human CD4; clone UMAB64 Clinisciences) and anti-FoxP3 (mouse monoclonal anti-human FOXP3; clone 236A/E7; AbCam), or anti-CD3, anti-CD8 and anti-PDL-1 were incubated for 30 min at room temperature.

Next, incubation with HRP Labeled Polymer mouse or rabbit antibodies was performed at room temperature for 15 min followed by fluorophores incubation for 10 min. At last, all slides were counterstained with DAPI for 5 min before scanning of whole slide and quantification using a proprietary digital pathology software developed by HalioDx. Results were tabulated and plotted using the Graphpad Prism software package

### Analysis of Gene Expression Change in Tumor Tissue During Treatment: Multiplex Immune Gene Expression

Nanostring nCounter technology was used to measure relative expression levels of immune genes within the tumor microenvironment on formalin fixed tumor tissue (thickness 10 μm). After extraction, Total RNA (300 ng) was assayed on an nCounter Digital Analyzer and hybridized to the Pan-cancer immune profiling panel, according to the manufacturer's instructions. The panel contains 770 genes, including key checkpoints, chemokines, cytokines, and associated control genes. The quality control and normalization of the data were done with the nSolver software package. The measured expression values were normalized to the geometric mean of the housekeeping gene expression levels with the lowest coefficient of variation (% CV). Statistical analysis was performed using the nSolver Advanced Analysis Module and the R software package. The Immunosign® was defined using a commercially available proprietary algorithm developed by HalioDx.

### Example 1A: Phase Ib Study and Results

### Study Population

Nine patients (4 females and 5 males) were enrolled in this study and treated with either 5×10⁶ pfu (DL1) or 5×10⁷ pfu (DL2) of TG4001 in combination with avelumab at 10 mg/kg according to the therapeutic regimen described above. Table 1 illustrates patient demographics and Characteristics at baseline.

TABLE 1
Patient demographics and Baseline Characteristics.

| | DL1 | | DL2 | | Overall | |
|---|---|---|---|---|---|---|
| | (n = 3) | | (n = 6) | | (n = 9) | |
| Mean age (range) years | 51.0 | (45-56) | 61.2 | (39-78) | 57.8 | (39-78) |
| Gender | | | | | | |
| Female | 2 | (66.7%) | 2 | (33.3%) | 4 | (44.4%) |
| Male | 1 | (33.3%) | 4 | (66.7%) | 5 | (55.6%) |
| Performance Status (ECOG) | | | | | | |
| 0 | 0 | (0.0%) | 4 | (66.7%) | 4 | (44.4%) |
| 1 | 3 | (100.0%) | 2 | (33.3%) | 5 | (55.6%) |
| Type of cancer | | | | | | |
| Anal | 2 | (66.7%) | 0 | (0.0%) | 2 | (22.2%) |
| Cervical | 0 | (0.0%) | 1 | (16.7%) | 1 | (11.1%) |
| Oropharyngeal | 1 | (33.3%) | 4 | (66.7%) | 5 | (55.6%) |
| Vaginal | 0 | (0.0%) | 1 | (16.7%) | 1 | (11.1%) |
| Histology | | | | | | |
| Adenocarcinoma | 0 | (0.0%) | 1 | (16.7%) | 1 | (11.1%) |
| Squamous cell carcinoma | 3 | (100.0%) | 5 | (83.3%) | 8 | (88.9%) |
| Number of prior CT lines | | | | | | |
| Median | 3.0 | | 1.5 | | 2.0 | |
| Range | 2-3 | | 1-2 | | 1-3 | |
| Locoregional relapse | 1 | (33.3%) | 2 | (33.3%) | 3 | (33.3%) |
| Distant Metastases | 3 | (100.0%) | 6 | (100.0%) | 9 | (100.0%) |

DL: dose level of TG4001.
CT: chemotherapy.

The baseline characteristics of the patients were as follows: median age 57.8 years (range 39-78) having various types of HPV-16-positive cancers (anal, cervical, oropharyngeal and vaginal) mainly of squamous cell carcinoma origin. At baseline, all patients showed distant metastases.

### Summary of Treatment Related Adverse Events

Safety was assessed through the reporting of adverse events (AEs) and by clinical laboratory tests, physical examinations, electrocardiograms (ECG) and vital signs at various time points. Adverse events and laboratory abnormalities were evaluated according to National cancer Institute Common Toxicity Criteria for Adverse Events (NCI-CTCAE). Toxicities were graded according to NCI-CTCAE, version 4.03. Treatment related adverse events data were summarized by dose level and reported in Table 2 below.

TABLE 2
Summary of treatment related adverse events.

| | | 5 × 10⁶ PFU | 5 × 10⁷ PFU |
|---|---|---|---|
| System Organ | | (N = 3) | (N = 6) |
| Class | Preferred Term | n (%) #AEs | n (%) #AEs |
| OVERALL | | 3 (100.0%) 23 | 6 (100.0%) 45 |
| Blood and | Anaemia | 2 (66.7%) *3 | 0 (0.0%) 0 |
| lymphatic system | Thrombocytopenia | 1 (33.3%) 1 | 0 (0.0%) 0 |
| disorders | | | |
| Endocrine | Hyperthyroidism | 1 (33.3%) 1 | 0 (0.0%) 0 |
| disorders | Hypothyroidism | 0 (0.0%) 0 | 1 (16.7%) 1 |
| Eye disorders | Xerophthalmia | 0 (0.0%) 0 | 1 (16.7%) 1 |
| Gastrointestinal | Diarrhoea | 0 (0.0%) 0 | 3 (50.0%) 3 |
| disorders | Dry mouth | 0 (0.0%) 0 | 1 (16.7%) 3 |
| | Mouth ulceration | 0 (0.0%) 0 | 1 (16.7%) 1 |
| | Nausea | 1 (33.3%) 1 | 0 (0.0%) 0 |
| | Stomatitis | 0 (0.0%) 0 | 1 (16.7%) 1 |
| | Tongue coated | 0 (0.0%) 0 | 1 (16.7%) 1 |
| | Vomiting | 1 (33.3%) 1 | 0 (0.0%) 0 |
| General | Asthenia | 2 (66.7%) 2 | 2 (33.3%) 2 |
| disorders and | Chills | 0 (0.0%) 0 | 1 (16.7%) 1 |
| administration | Fatigue | 0 (0.0%) 0 | 1 (16.7%) 1 |
| site conditions | Injection site | 1 (33.3%) 3 | 3 (50.0%) 5 |
| | erythema | | |
| | Injection site | 0 (0.0%) 0 | 2 (33.3%) 4 |
| | oedema | | |
| | Injection site pain | 0 (0.0%) 0 | 1 (16.7%) 2 |
| | Injection site | 1 (33.3%) 1 | 0 (0.0%) 0 |
| | reaction | | |
| | Mucosal | 0 (0.0%) 0 | 1 (16.7%) 1 |
| | inflammation | | |
| | Pyrexia | 1 (33.3%) 5 | 2 (33.3%) 4 |
| Infections and | Angular cheilitis | 0 (0.0%) 0 | 1 (16.7%) 1 |
| infestations | | | |
| Investigations | Aspartate | 1 (33.3%) 2 | 0 (0.0%) 0 |
| | aminotransferase | | |
| | increased | | |
| | Blood thyroid | 0 (0.0%) 0 | 1 (16.7%) 1 |
| | stimulating | | |
| | hormone increased | | |
| Metabolism and | Hyperkalaemia | 0 (0.0%) 0 | 1 (16.7%) 1 |
| nutrition disorders | | | |
| Musculoskeletal | Arthralgia | 0 (0.0%) 0 | 1 (16.7%) 1 |
| and connective | Myalgia | 1 (33.3%) 1 | 0 (0.0%) 0 |
| tissue disorders | Neck pain | 0 (0.0%) 0 | 1 (16.7%) 1 |
| | Sjogren's syndrome | 0 (0.0%) 0 | 1 (16.7%) 1 |
| Nervous system | Peripheral sensory | 0 (0.0%) 0 | 1 (16.7%) 1 |
| disorders | neuropathy | | |
| Renal and urinary | Proteinuria | 0 (0.0%) 0 | 1 (16.7%) 1 |
| disorders | | | |
| Respiratory, | Dyspnoea | 0 (0.0%) 0 | 1 (16.7%) 1 |
| thoracic and | | | |
| mediastinal | | | |
| disorders | | | |
| Skin and | Pruritus | 0 (0.0%) 0 | 1 (16.7%) 1 |
| subcutaneous | Rash | 1 (33.3%) 1 | 1 (16.7%) 1 |
| tissue | Rash macular | 0 (0.0%) 0 | 1 (16.7%) 2 |
| disorders | Rash maculo- | 0 (0.0%) 0 | 1 (16.7%) 1 |
| | papular | | |
| | Rash papular | 1 (33.3%) 1 | 0 (0.0%) 0 |

N: number of patients.
%: percentage of patients.
#AEs: Number of adverse events.
*presence of two grade 3 adverse event in one patient.

As illustrated in Table 2, all patients experienced at least one AE. Specifically, a total of 68 adverse events in 9 patients have been observed: 23 in DL1 and 45 in DL2. the TG4001 and avelumab combination treatment was well tolerated. Indeed, no treatment-related serious adverse event (SAE) was observed and only two grade 3 events were observed in one DL1-treated patient (5×10⁶ PFU).

### Changes in Tumor Size

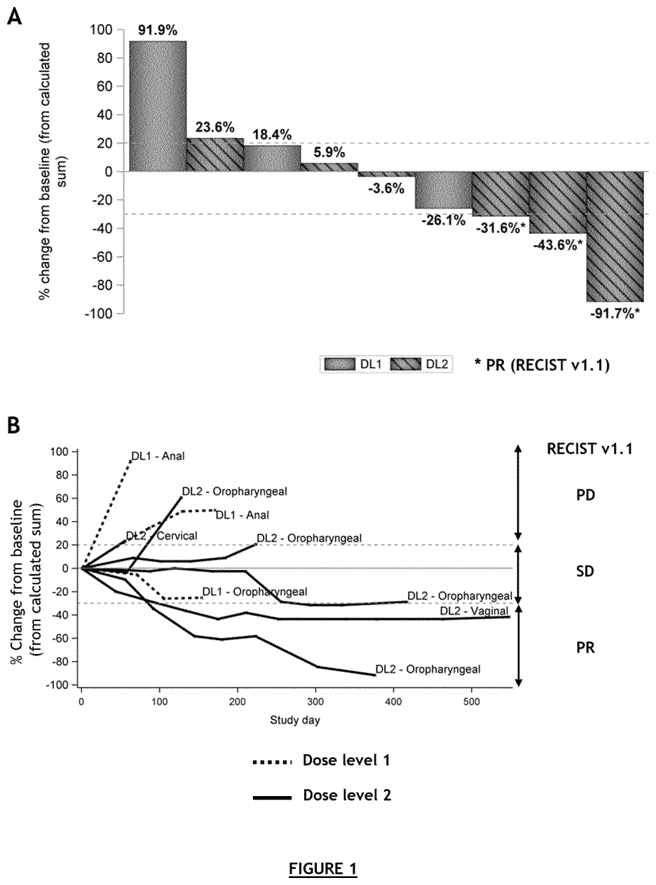

Changes in tumor size during combination treatment are presented in FIGS. 1A and 1B. As illustrated, most of DL2-treated patients experimented a reduction or a stabilization in tumor size.

Partial response (PR), stable disease (SD) and progressive disease (PD) numbers and percentages in the course of the clinical study, assessed under RECIST 1.1 criteria, are also presented in Table 3 below.

TABLE 3
Numbers and percentages of partial response (PR),
stable disease (SD) and progressive disease (PD).

| | Partial | | stable | progressive |
|---|---|---|---|---|
| | response (PR) | | disease (SD) | disease (PD) |
| | N (%) | | N (%) | N (%) |
| DL1 5 × 10⁶ PFU | 0 | (0%) | 1 (33%) | 2 (67%) |
| n = 3 | | | | |
| DL2 - 5 × 10⁷ PFU | 3 | (50%) | 2 (33%) | 1 (17%) |
| n = 6 | | | | |
| Total | 3 | (33%) | 3 (33%) | 3 (33%) |
| n = 9 | | | | |

Results of Table 3 and FIGS. 1A and 1B show that the combination treatment is able to stabilize disease or to induce partial responses in two third of all patients. The efficiency seems better at higher dose DL2 of TG4001 (5×10⁷ PFU), with stable disease or partial response in about 83% of patients.

### Immune Data

### Specific T Cell Responses Against HPV

Four patients were evaluable for ELISPOT responses against HPV-16 E6 and E7 at day 43. ELISPOT responses are presented in Table 4 below.

TABLE 4
ELISPOT responses against HPV-16 E6 and E7 at day 43.

| | E6 | E7 |
|---|---|---|
| Lines are individual | ++ | + |
| patients | n/a | ++ |
| | + | = |
| | n/a | − |

n/a: not applicable; −: 0 spot observed; =: <10 spots observed; +: 10-100 spots observed; ++: >100 spots observed.

Table 4 shows that 3 out of 4 patients evaluable for ELISPOT had E6 or E7 reactive T cells at day 43.

### Changes in TILs Under Treatment

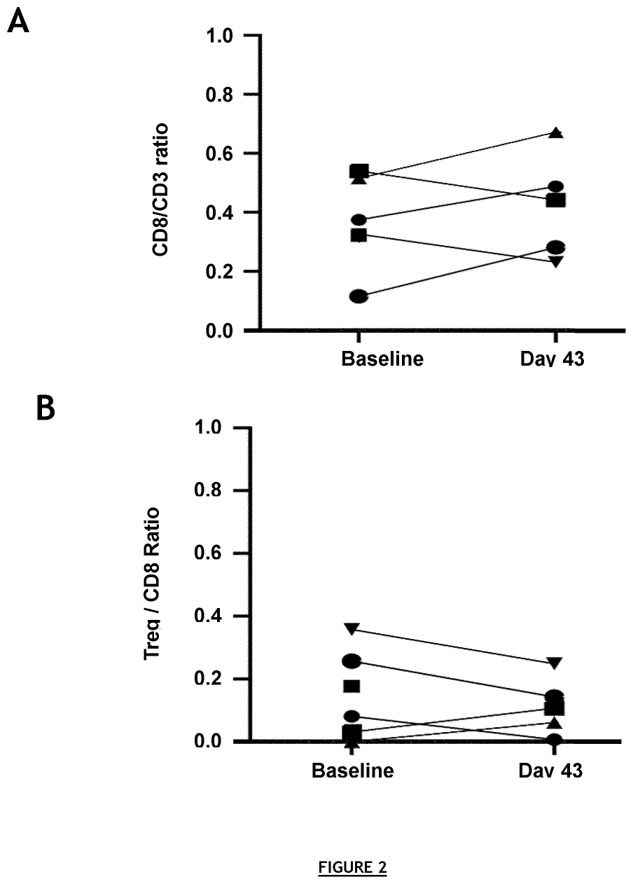

CD8/CD3 ratio and Treg (CD4 FoxP3)/CD8 ratio at baseline and at day 43 are presented in FIGS. 2A and 2B, showing that treatment period was associated in overall increase of CD8/CD3 ratio infiltrates and decrease of Treg (CD4 FoxP3)/CD8 ratio suggesting a more favorable immune profile (enhancement of the immunostimulator CD8 T cells and decrease of the immunosuppressor Treg).

### PD-L1 Expression on TILs and Tumor Cells Under Treatment

PD-L1 expression was evaluated on TILs and tumor cells at baseline and at day 43 (and also at day 85 for one patient) in 7 patients, 5 of which were evaluable at both baseline and day 43. Results are presented in Table 5 below.

TABLE 5
Percentage of PD-L1+ tumor cells measured
by immunohistochemistry (IHC) and immune cells
score at baseline, day 43, and day 85 (1 patient).

| | | % of PD-L1+ | Immune cells |
|---|---|---|---|
| Primary Cancer | Time point | tumor cells | score |
| vaginal | Baseline Biopsy | 35 | 0 |
| | Day 43 Biopsy | NA | 0 |
| Anal* | Baseline Biopsy | 50 | 1 |
| | Day 43 Biopsy | 40 | 0 |
| anal | Baseline Biopsy | 0 | 3 |
| | Day 43 Biopsy | NA | NA |
| Oropharyngeal* | Baseline Biopsy | 80 | 2 |
| | Day 43 Biopsy | 90 | 2 |
| | Day 85 Biopsy | 80 | 1 |
| Cervical* | Baseline Biopsy | 40 | 1 |
| | Day 43 Biopsy | 80 | 2 |
| Oropharyngeal* | Baseline Biopsy | 15-20 | 1 |
| | Day 43 Biopsy | 35 | 3 |
| Oropharyngeal* | Baseline Biopsy | 20 | 1 |
| | Day 43 Biopsy | 40 | 1 |

NA: not applicable.
*Patients evaluable at both baseline and day 43.

Results of Table 5 show that 4 out of 5 patients evaluable at both baseline and day 43 had a significant increase of PD-L1 expression in tumor cells at day 43, which is expected to correlate with an increase propensity to respond to immunotherapy treatment.

### Analysis of Gene Expression Change in Tumor Tissue During Treatment

To explore gene expression changes in tumor tissue induced by therapy, the expression of a panel of 770 genes related to immune response was assessed at baseline and after treatment (day 43). In particular, expression of gene signatures previously described as Immunosign® 15 and Immunosign® 21 were studied (Galon et al., Immunity (2013) 9(1): 11-26; Marabelle et al., Society for Immunotherapy of Cancer (SITC) 32^{nd} Annual Meeting [symbol] Pre-Conference Programs (SITC 2017) on Nov. 8-12, 2017 at the Gaylord National Hotel [symbol] Convention Center in National Harbor, Maryland. Poster P250).

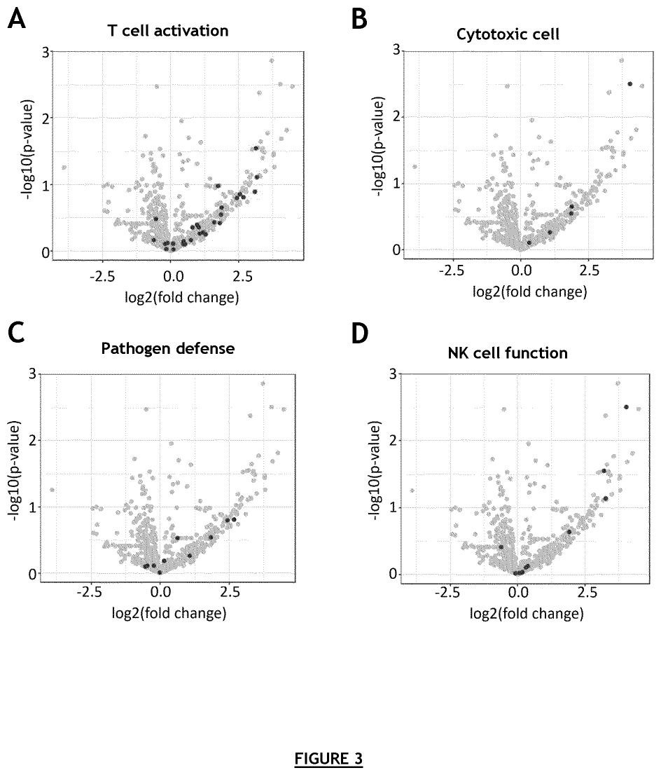

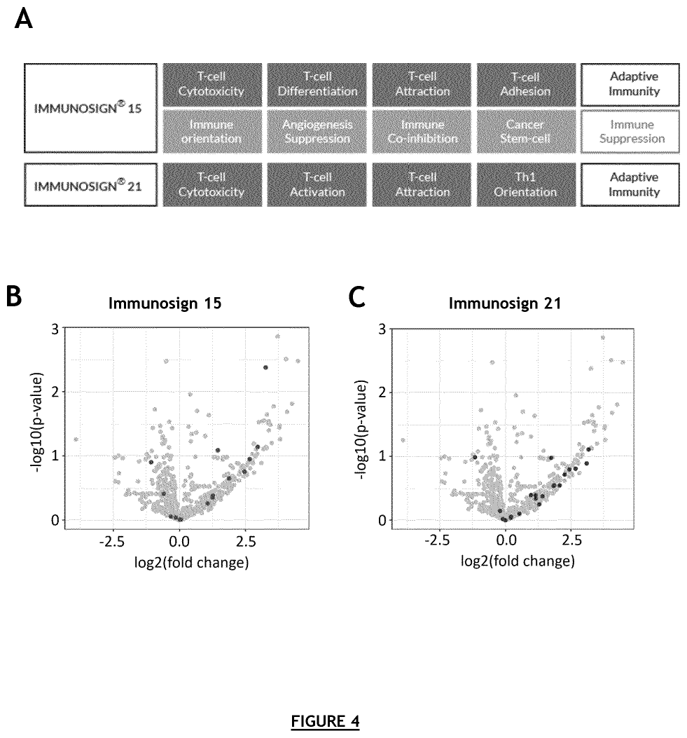

Volcano plots of changes in gene expression and pathways identified as overexpressed post vs. pre-treatment are presented in FIGS. 3 and 4. Black dots represent the relevant genes involved in the indicated mechanism (e.g. T cell activation in FIG. 3A, etc), the level of expression correlating with the position in the log scale (overexpression at the right of the 0 value and underexpression at the left of the 0 value).

FIG. 3 shows that pathways related to viral vaccine response (pathogen defense, see FIG. 3C) and priming of antitumor immunity (T cell activation, cytotoxic cell, and NK cell function, see FIGS. 3A, 3B, and 3D) were overexpressed during treatment.

Moreover, FIG. 4A presents a description of gene categories included in gene signatures previously described as Immunosign® 15 and Immunosign® 21, and FIGS. 4B and 4C show that many genes of Immunosign® 15 and Immunosign® 21 signatures were overexpressed during treatment.

The observed gene expression changes are consistent with a priming of innate and adaptive immunity and shift to a “hotter” tumor profile which is more consistently associated with an improved clinical outcome for the patient.

### Case Study on Patient 0101006

More detailed data is presented for patient 0101006, suffering from cervical cancer.

Changes in immune infiltrates:

Patient 0101006 presented with a tumor with low level of infiltration, moderate expression of PD-L1 on tumor cells and infiltrating immune cells, and, low spatial colocalization between CD8 cells and PD-L1 expressing tumor cells. All these features are consistent with a cold tumor at baseline.

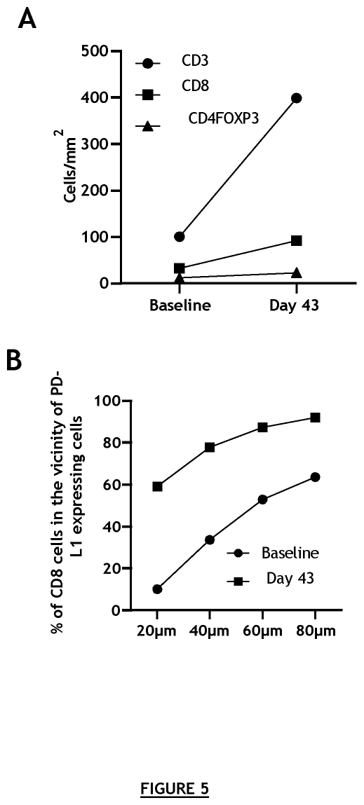

Changes in immune infiltrates are presented in FIG. 5, and PD-L1 expression on TILs and tumor cells at baseline and at day 43 is presented in Table 6 below.

TABLE 6
Percentage of PD-L1+ tumor cells measured
by immunohistochemistry (IHC) and immune cells
score at baseline and day 43 in patient 0101006.

| Time point | % of PD-L1+ tumor cells | Immune cells score |
|---|---|---|
| Baseline Biopsy | 40 | 1 |
| Day 43 Biopsy | 80 | 2 |

FIG. 5 and Table 6 show that, at day 43, the tumor was significantly more infiltrated with more than a 4-fold increase in CD3 infiltration, a 3-fold increase in CD8 infiltration, and a doubling of tumor and immune expressing PD-L1. No significant change was observed on the level of infiltration by immune suppressive cells during treatment.

Furthermore, digital pathology analysis of sample demonstrates that infiltrated CD8 are clustered around PD-L1 positive tumor cells (FIG. 5B), suggesting a more favorable profile for response to checkpoint blockade.

Changes in gene expression:

Gene expression profile in tumor tissue of patient also revealed significant changes over the treatment period as shown on the color map of 770 immune related genes (data not shown).

Analysis of gene expression changes further shows a strong increase in expression of genes related to antigen processing and presentation (FIG. 6A), response to virus (FIG. 6B) and expression of Toll-like receptors (FIG. 6C). The activation of these pathways is consistent with the development of an adaptive response against the viral vaccine.

Also, 9 genes of the Immunosign® 21 signature were strongly overexpressed (FIG. 6E) suggesting a shift from an immune excluded phenotype to a “hot” tumor likely to benefit from immunotherapeutic intervention.

### Conclusions

Preliminary results of NCT03260023 phase Ib shows that:
The combination of TG4001 and avelumab is safe and well tolerated for both dose levels of TG4001 studied in patients with HPV-positive cancers having received multiple previous lines of treatment.
The combination provides promising efficacy signals at DL2 and is being evaluated in the ongoing phase II part.
The treatment is associated with changes in tumor microenvironment that are likely to change the course of the pathology by shifting tumor from a cold state to a hotter immune status even in heavily pretreated patients.
This may be particularly useful in patient with “immune-excluded” tumor phenotype as shown on the individual study case.

These results suggest that a combination of (a) a poxvirus vector encoding at least human papillomavirus (HPV) E6 and E7 polypeptides and an immunostimulatory cytokine and (b) an anti-PD-L1 antibody is safe, well tolerated and effective (at least additive efficacy) in the treatment of patients with HPV-positive cancers.

### Example 18: Phase II Study and Pooled Interim Analysis

### Patients/Disease Characteristics

For the phase II, TG4001 was administered at DL2. DL2 was the recommended phase II dose after completion of phase IB and supported by the results showing that the combination of TG4001 and avelumab is safe and regarding nature and severity of reported AEs no notable difference could be observed between DL1 and DL2. For the interim analysis as planned per protocol, 25 patients were enrolled in this study out of which 3 were not evaluable for tumor response. Of the 22 evaluable patients 11 patients (50%) presented anal cancer, 4 patients (18.2%) presented cervical cancer, 4 patients (18.2%) presented vaginal/vulvar cancer and 3 patients (13.6%) presented oropharyngeal cancer. Before the inclusion hold for the interim analysis, additional 7 patients were enrolled out of which 1 patient was not evaluable for tumor response. Of the 6 evaluable patients, 4 patients (66.6%) presented anal cancer, and 1 patient each presented oropharyngeal cancer (16.6%) and cervical cancer (16.6), respectively.

For subgroup analysis, patients treated in the phase Ib part with DL2 of TG4001 (N=6) have been pooled with patients treated in the phase II part (N=28 as discussed just above). Of the 6 patients treated in phase Ib, 4 patients (66.6%) presented oropharyngeal cancer, and 1 patient each presented vaginal cancer (16.6%) and cervical cancer (16.6), respectively. Of the pooled data set (N=34), 15 patients (44.1%) presented anal cancer, 8 patients (23.5%) presented oropharyngeal cancer, 6 patients (17.6%) presented cervical cancer and 5 patients (14.7%) presented vaginal /vulvar cancer. An ORR of 20.6% has been observed.

The pooled patient population was stratified for the presence of liver metastases (w/o versus w as shown in Table 7, for the disease characteristics (Table 8) and for prior chemotherapy treatment (Table 9).

Table 7 provides an overview of the gender and performance status (PS) for the subgroups of patients with and without liver metastases. Specifically, the subgroup of patients without liver metastases represents 23 patients (non-hepatic patients) whereas the subgroup of patients with liver metastases accounts 11 patients (hepatic patients)

TABLE 7
TG4001.12 stratification for the absence (non-hepatic patients)
or the presence (hepatic patients) of liver metastases.

| | | Non Hepatic | Hepatic | |
|---|---|---|---|---|
| | | Patients | Patients | Overall |
| | | (N = 23) | (N = 11) | (N = 34) |
| Gender | Male | 9 pts (26.5%) | 3 pts (8.8%) | 12 pts (35.3%) |
| | Female | 14 pts (41.1%) | 8 pts (23.5%) | 22 pts (64.7%) |
| PS | 0 | 7 pts (20.6%) | 7 pts (20.6%) | 14 pts (41.1%) |
| | 1 | 16 pts (47.0%) | 4 pts (11.8%) | 20 pts (58.8%) |

PS: performance status.
pts: patients.

Concerning the disease characteristics in the pooled data set (N=34), as shown in Table 8, 15 patients presented anal cancer, 8 patients presented oropharyngeal cancer, 6 patients presented cervical cancer and 5 patients presented vaginal/vulvar cancer. An ORR of 20.6% has been observed.

TABLE 8
Stratification by disease characteristics
of the pooled patient population.

| | | Non Hepatic | Hepatic | |
|---|---|---|---|---|
| | | Patients | Patient | Overall |
| | | (N = 23) | (N = 11) | (N = 34) |
| Primary | Anal | 7 pts (20.6%) | 8 pts (23.5%) | 15 pts (44.1%) |
| tumor | Cervical | 5 pts (14.7%) | 1 pts (2.9%) | 6 pts (17.6%) |
| | Oropharynx | 8 pts (23.5%) | — | 8 pts (23.5%) |
| | Vaginal | 2 pts (5.9%) | 2 pts (5.9%) | 4 pts (11.8%) |
| | Vulvar | 1 pt (2.9%) | — | 1 pt (2.9%) |
| Number | 1 organ | 9 pts (26.5%) | 3 pts (8.8%) | 12 pts (35.3%) |
| organs | 2 organs | 10 pts (29.4%) | 3 pts (8.8%) | 13 pts (38.2%) |
| affected | ≥3 organs | 4 pts (11.8%) | 5 pts (14.7%) | 9 pts (26.5%) |

| | | | |
|---|---|---|---|
| Lymph node | 15 pts (44.1%) | 3 pts (8.8%) | 18 pts (52.9%) |
| Lung | 10 pts (29.4%) | 3 pts (8.8%) | 13 pts (38.2%) |
| Bone | 2 pts (5.9%) | 2 pts (5.9%) | 4 pts (11.8%) |
| Primary tumor/other | 13 pts (38.2%) | 5 pts (14.7%) | 18 pts (52.9%) |

pts: patients.

TABLE 9
stratification by prior chemotherapy regimens or no
prior treatment of the polled patient population.

| | | Non Hepatic | Hepatic | |
|---|---|---|---|---|
| | | Patients | Patient | Overall |
| | | (N = 23) | (N = 11) | (N = 34) |
| Prior | No | 10 pts (29.4%) | 4 pts (11.8%) | 14 pts (41.1%) |
| chemotherapy | Yes | 13 pts (38.2%) | 7 pts (20.6%) | 20 pts (58.8%) |
| for local/ | | | | |
| locally | | | | |
| advanced | | | | |
| disease | | | | |
| Prior | No | 4 pts (11.8%) | — | 4 pts (11.8%) |
| chemotherapy | 1 line | 14 pts (41.2%) | 5 pts (14.7%) | 19 pts (55.9%) |
| for recurrent/ | ≥2 | 5 pts (14.7%) | 6 pts (17.6%) | 11 pts (32.3%) |
| metastatic | lines | | | |
| disease | | | | |

pts: patients.

### Correlation of Patients/Disease Characteristics with ORR and PFS

Based on the above stratifications, correlation of each patients/disease characteristic with objective response rate (ORR) or progression-free survival (PFS) was assessed.

Correlations of each patients/disease characteristic with ORR and PFS are presented in FIGS. 7 and 8, respectively.

With respect to ORR, only one disease characteristic (boxed in FIG. 7) was found to be significantly correlated with a worse ORR: the presence of liver metastases (see FIG. 7, showing an OR of 100, a 95% interval of 5-100, and a p-value of 0.012).

With respect to PFS, three characteristics were found as significantly correlated to a worse or better PFS (they are boxed in FIG. 8):
the presence of liver metastasis, significantly correlated to a worse PFS (see FIG. 8, showing a HR of 4.390, a 95% interval of 1.728-11.152, and a p-value of 0.002),
anal cancer, significantly correlated to a worse PFS (see FIG. 8, showing a HR of 3.214, a 95% interval of 1.269-8.143, and a p-value of 0.014), and
the presence of lymph node metastasis, significantly correlated to a better PFS (see FIG. 8, showing a HR of 0.388, a 95% interval of 0.151-0.998, and a p-value of 0.049).

With respect to the association of anal cancer with lower PFS, it should be noted that this association may be explained indirectly by higher prevalence of liver metastasis in anal cancer patients. Indeed, responses have been documented in patients with anal cancer not presenting metastases in the liver but in other organs.

Moreover, with respect to PFS, while not found significant (p=0.073) with the number of patients analyzed in the pooled interim analysis, a tendency towards correlation of genital (vulvar/vaginal) cancer and better PFS is observed.

The only characteristic significantly correlated to both ORR and PFS was thus the presence of liver metastases, significantly correlated to worse ORR and PFS.

This stratification has thus been more thoroughly studied.

Table 10 below further shows the distribution of patients with or without liver metastases depending on efficacy parameters (RECIST1.1 response, stable disease at 12 weeks, progression before/at 12 weeks, and median PFS), and shows that, when treated with the combination treatment, patients without liver metastases have higher response and stable disease at 12 weeks, lower progression before/at 12 weeks and higher median PFS than patients with liver metastases.

TABLE 10
Distribution of patients with or without liver
metastases depending on efficacy parameters.

| | Patients without | | Patients with | | | |
|---|---|---|---|---|---|---|
| | liver metastases | | liver metastases | | Overall | |
| Efficacy parameters | (N = 23) | | (N = 11) | | (N = 34) | |
| Response | 7 | (30.4%) | 0 | (0%) | 7 | (20.6%) |
| (RECIST1.1) | | | | | | |
| Stable disease | 6 | (26.1%) | 1* | (9.1%) | 7 | (20.6%) |
| at 12 weeks | | | | | | |
| Progression ≤12 | 10 | (43.5%) | 10 | (90.9%) | 20 | (58.8%) |
| weeks | | | | | | |
| Median PFS in | 5.6 | [1.5-20.7] | 1.4 | [1.1-1.5] | 2.9 | [1.4-5.6] |
| months [range] | | | | | | |

*Patient presented single hepatic lesion of 12 mm.

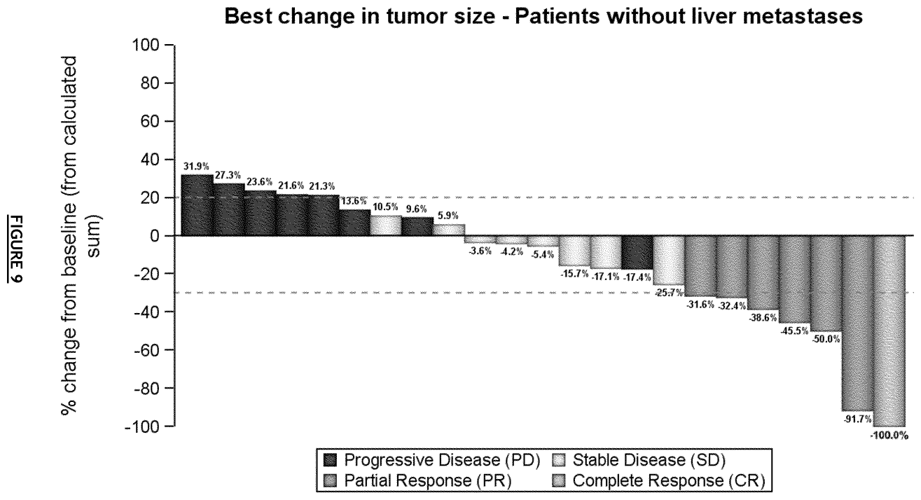

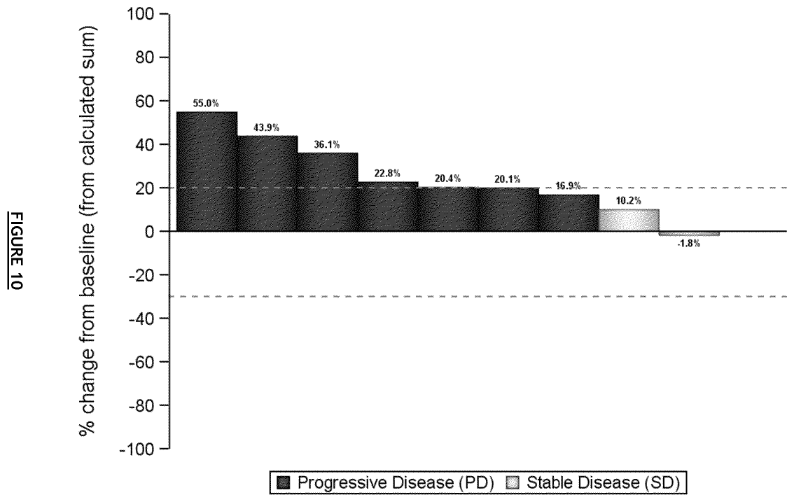

FIG. 9 presents a graphical representation of best change in tumor size in the 23 patients from pooled phase Ib and phase II interim results without liver metastases, FIG. 10 presenting the same graphical representation for 9 patients from pooled phase Ib and phase II interim results with liver metastases.

Table 11 below shows the distribution of patients with or without liver metastases depending on the type of primary tumor (anal, oropharyngeal, cervical, vulvar/vaginal), and shows that, no matter the primary tumor, patients without liver metastases have a higher response than patients with liver metastases (no response observed in these patient, no matter the primary tumor). Table 11 also shows a high response rate for vulvar/vaginal cancer patients without liver metastases (66.7%), although this high rate is to be taken with precaution due to the low number of analyzed patients (only 3).

TABLE 11
Distribution of patients with or without liver metastases
depending on the location of primary tumor.

| Response according | Patients | Patients | | |
|---|---|---|---|---|
| to primary tumor | without liver | with liver | | |
| (responders/total | metastases | metastases | Overall | |
| number of patients %) | (N = 23) | (N = 11) | (N = 34) | |
| Anal | 2/7 (28.6%) | 0/8 (0%) | 2/15 | (13.3%) |
| Oropharyngeal | 2/8 (25.0%) | — | 2/8 | (25.0%) |
| Cervical | 1/5 (20.0%) | 0/1 (0%) | 1/6 | (16.7%) |
| Vulvar/vaginal | 2/3 (66.7%) | 0/2 (0%) | 2/5 | (40%) |

### Immune Data

### Specific T Cell Responses Against HPV

Eleven patients were evaluable for ELISPOT responses against HPV-16 E6 and E7 at day 43. Table 12 illustrates that 7 out of 11 patients had detectable response by ex vivo ELISPOT after vaccination. None of the patients had a preexisting response in this experimental setting prior to vaccination. ELISPOT responses are presented in Table 12 below.

TABLE 12
Patients with antigen reactive T-cells against HPV E6
and E7 as assessed by ELISPOT at baseline and Day 43.

| | |
|---|---|
| Prior to vaccination | After Vaccination |

| | E6 | E7 | E6 | E7 |
|---|---|---|---|---|
| Lines are | 0 | 0 | + | + |
| individual | 0 | 0 | ++ | 0 |
| patients | 0 | 0 | ++ | ++ |
| | 0 | 0 | 0 | + |
| | 0 | 0 | + | + |
| | 0 | 0 | + | 0 |
| | 0 | 0 | 0 | ++ |
| | 0 | 0 | 0 | 0 |
| | 0 | 0 | 0 | 0 |
| | 0 | 0 | 0 | 0 |
| | 0 | 0 | 0 | 0 |

n/a: not applicable; −: 0 No response; +: >Background + 2 SD, ++: >Background + 3 SD spots observed.

### Immune Infiltrate

Observations made during the phase 1 of the study were confirmed in the phase 2 with increases of genes related to the Immunosign® signatures and increase in infiltrates of CD3 positive cells.

### Transcriptomic Analysis

Transcriptomic analysis of tumors collected from patients with or without liver tumor metastasis was carried out as described in Material and Methods.

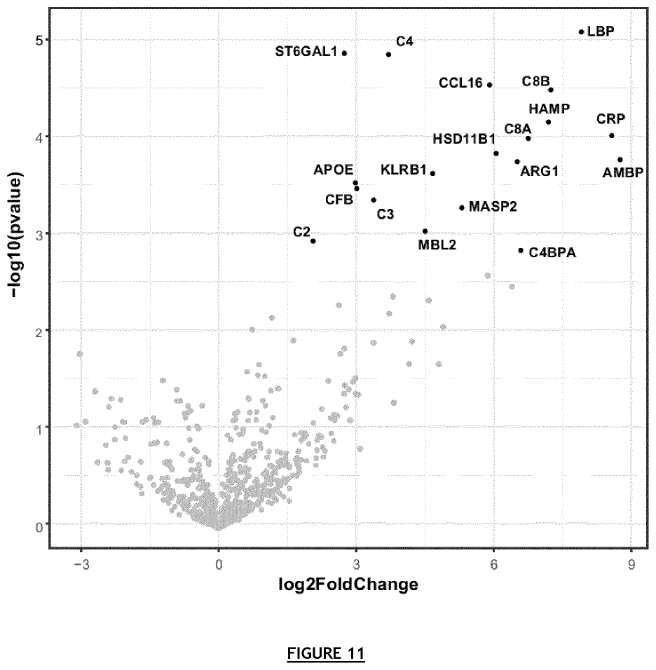

The inventors were able to show gene expression variations in liver metastases. Notably, as illustrated in Table 13 and FIG. 11, ST6GAL1 (p<0.001) and HAMP (p<0.0001) genes were overexpressed (Log 2 change of 2.74 and 7.15, respectively). Genes associated with the complement pathway were over-represented including C8A (Log 2 change of 6.71; p<0.001), C8B (Log 2 change of 7.25; p<0.001), C3 (Log 2 change of 3.41; p<0.001), C6 (Log 2 change of 5.87; p<0.001) and C2 (Log 2 change of 2.06; p<0.001).

ST6GAL1 is associated with aggressiveness in many cancers and HAMP is known to regulate immune cell activity through alteration of iron metabolism.

The effect of the complement on cancer immunity remains controversial, high expression of the complement pathway has been shown to exert a cytotoxic action toward immune cells including effector CD4 and CD8. This is of interest given that the liver is the main site of synthesis and regulation of complement factors.

Additionally, cytokines associated with inflammation are also represented and may contribute to the creation of an immunosuppressive tumor environment. This immunosuppression is deleterious for effector immune cells and can drive immune escape of the tumor and progression of the disease. These unique transcriptomic features are thus consistent with resistance to immune-intervention in patients with hepatic metastasis.

TABLE 13
Genes differentially expressed between patient
with or without liver metastatis

| | Log2 fold change | P-value | FDR |
|---|---|---|---|
| ST6GAL1 | 2.7400 | 0.0000 | 0.0226 |
| CCL16 | 5.9100 | 0.0000 | 0.0326 |
| C8B | 7.2500 | 0.0000 | 0.0326 |
| HAMP | 7.1500 | 0.0001 | 0.0560 |
| CRP | 8.5800 | 0.0001 | 0.0634 |
| C8A | 6.7100 | 0.0001 | 0.0634 |
| HSD11B1 | 6.0800 | 0.0001 | 0.0753 |
| ARG1 | 6.4800 | 0.0002 | 0.0753 |
| AMBP | 8.7600 | 0.0002 | 0.0753 |
| KLRB1 | 4.6800 | 0.0002 | 0.0942 |
| APOE | 3.0000 | 0.0003 | 0.1090 |
| CFB | 3.0300 | 0.0003 | 0.1160 |
| C3 | 3.4100 | 0.0004 | 0.1420 |
| MASP2 | 5.3400 | 0.0005 | 0.1600 |
| MBL2 | 4.4800 | 0.0008 | 0.2550 |
| C2 | 2.0600 | 0.0011 | 0.3170 |
| C4BPA | 6.5800 | 0.0014 | 0.3630 |
| C6 | 5.8700 | 0.0025 | 0.6250 |
| C9 | 6.4000 | 0.0032 | 0.7770 |
| DPP4 | 3.8000 | 0.0040 | 0.9350 |
| CCL14 | 4.5900 | 0.0044 | 0.9840 |
| IHH | 2.6300 | 0.0050 | 1.0000 |
| CD180 | 3.7200 | 0.0061 | 1.0000 |
| MERTK | 1.1600 | 0.0067 | 1.0000 |
| SAA1 | 4.9000 | 0.0084 | 1.0000 |
| PVR | 0.7310 | 0.0089 | 1.0000 |
| SERPING1 | 1.6300 | 0.0115 | 1.0000 |
| C8G | 4.2200 | 0.0118 | 1.0000 |
| CARD9 | 3.3800 | 0.0122 | 1.0000 |
| MARCO | 2.7400 | 0.0139 | 1.0000 |
| CFI | 2.6500 | 0.0159 | 1.0000 |
| CCR6 | −3.0300 | 0.0159 | 1.0000 |
| CCL21 | 4.1500 | 0.0202 | 1.0000 |
| C7 | 4.8000 | 0.0202 | 1.0000 |
| CXCL16 | 0.8840 | 0.0208 | 1.0000 |
| C1QBP | 0.6210 | 0.0246 | 1.0000 |
| CD59 | 0.8590 | 0.0267 | 1.0000 |
| CTSL | 1.0100 | 0.0274 | 1.0000 |
| CD1D | 2.9800 | 0.0281 | 1.0000 |
| TNFSF10 | −1.2200 | 0.0299 | 1.0000 |
| SH2D1A | 2.3900 | 0.0301 | 1.0000 |
| TNFSF14 | 2.9300 | 0.0306 | 1.0000 |
| CCL15 | 2.7500 | 0.0335 | 1.0000 |
| KIR Inhibiting Subgroup 1 | 1.3100 | 0.0364 | 1.0000 |
| IFNL1 | 1.2800 | 0.0364 | 1.0000 |
| POU2F2 | 2.8400 | 0.0369 | 1.0000 |
| ITGA2 | −0.9070 | 0.0377 | 1.0000 |
| C1S | 1.1400 | 0.0386 | 1.0000 |
| CXCL10 | −2.7000 | 0.0388 | 1.0000 |
| ABCB1 | 2.7300 | 0.0409 | 1.0000 |
| IL6R | 2.9800 | 0.0411 | 1.0000 |

In conclusion, clinical observations of patients have highlighted liver metastasis as a determinant of response to treatment and clinical outcome. Genomic data revealed that tumor liver metastasis were characterized by the expression of genes and pathways associated with downregulation of the immune system or the aggressiveness of the tumor.

### Conclusion

In conclusion, pooled interim analysis of phase Ib and phase II shows that the presence of liver metastases is associated with reduced ORR and shorter PFS. Indeed, by stratifying the patients for the presence or absence of liver metastases, an ORR of 30.4% has been observed in the subgroup of patients without liver metastases (N=23) compared to 0% in the subgroup of patients with liver metastases (N=11). Similarly, the median PFS of patients without liver metastases (N=23) is 5.6, while the median PFS of patients with liver metastases (N=11) is only 1.4.

Moreover, pooled interim analysis of phase Ib and phase II also shows that anal cancer is associated with lower PFS due to higher prevalence of liver metastasis in anal cancer patients, while vulvar/vaginal cancer shows a tendency towards higher PFS.

Finally, lymph node metastasis is associated with better PFS.

1. A method of for treating an HPV-positive cancer or HPV-positive precancerous intraepithelial lesions in a subject in need thereof, comprising administering to said subject a combination of:
a) a poxvirus vector encoding at least human papillomavirus (HPV) E6 and E7 polypeptides and an immunostimulatory cytokine, and
b) an anti-PD-L1 antibody or antigen-binding fragment thereof,
wherein a first administration of said poxvirus is performed 5 to 10 days before the first administration of said anti-PD-L1 antibody, and subsequent administrations of said poxvirus and anti-PD-L1 antibody are performed.

2. The method of claim 1, wherein said poxvirus is a vaccinia virus.

3. The method of claim 1, wherein said poxvirus encodes membrane anchored HPV-16 non-oncogenic E6 and E7 polypeptides and human interleukin 2 (IL-2).

4. The method of claim 1, wherein said anti-PD-L1 antibody mediates antibody-dependent cell-mediated cytotoxicity (ADCC).

5. The method of claim 1, wherein said anti-PD-L1 antibody or antigen-binding fragment thereof comprises a heavy chain, which comprises three complementarity determining regions having amino acid sequences of SEQ ID Nos: 1, 2 and 3, and a light chain, which comprises three complementarity determining regions having amino acid sequences of SEQ ID Nos: 4, 5 and 6.

6. The method of claim 1, wherein said anti-PD-L1 antibody is avelumab.

7. The method of claim 1, wherein said HPV-positive cancer is HPV-positive oropharyngeal, cervical, vaginal, anal, vulvar, penile, mucosal, or non-melanoma skin cancer, or said precancerous intraepithelial lesions are cervical intraepithelial neoplasia (CIN) grade 2 or 3 or vulvar intraepithelial neoplasia (VIN) grade 2 or 3.

8. The method of claim 7, wherein said cancer is HPV-16 positive.

9. The method of claim 1, wherein said HPV-positive cancer is HPV-positive cancer without liver metastasis.

10. The method of claim 7, wherein said HPV-positive cancer is recurrent or metastatic HPV-positive cancer.

11. The method of claim 10, wherein said HPV-positive metastatic cancer is HPV-positive metastatic cancer without liver metastasis.

12. The method of claim 1, wherein each administration of said poxvirus is performed at a dose of 3×10⁷ to 7×10⁷ pfu.

13. The method of claim 1, wherein the combination is administered with the following administration scheme:
a) a first dose of 3×10⁷ to 7×10⁷ pfu of said poxvirus is administered subcutaneously, and followed until disease progression by subsequent poxviruses doses of 3×10⁷ to 7×10⁷ pfu administered subcutaneously:
on a weekly basis for 6 weeks,
once every 2 weeks up to month 6, and
every 12 weeks, for the next poxviruses doses;
b) a first dose of about 10 mg/kg or about 800 mg of anti-PD-L1 antibody is administered intravenously 5 to 10 days after the first poxvirus dose, followed by subsequent anti-PD-L1 antibody doses of about 10 mg/kg or about 800 mg, which are administered intravenously every 2 weeks until disease progression.

14. The method of claim 1, wherein:
a) said poxvirus is an MVA virus encoding membrane anchored HPV-16 non-oncogenic E6 and E7 polypeptides and human IL-2,
b) said anti-PD-L1 antibody is avelumab, and
c) said poxvirus and anti-PD-L1 antibody are administered with the following administration scheme:
i) the MVA virus encoding membrane anchored HPV-16 non-oncogenic E6 and E7 polypeptides and human IL-2 is administered subcutaneously at a dose of 5×10⁷ pfu on a weekly basis for 6 weeks, then once every 2 weeks up to Month 6, and every 12 weeks thereafter until disease progression,
ii) avelumab is administered by intravenous infusion at a dose of about 10 mg/kg or about 800 mg every 2 weeks starting from Day 8 until disease progression.

15. The method of claim 1, wherein said combination induces:
an increase in an immune response against HPV16 E6 and E7 polypeptides;
within the tumor:
an increase in immune cell infiltrates,
decrease in regulatory CD4 T cells;
an increase of PD-L1 expression on tumor cells;
in the blood circulation:
an increase in CD8 T cells;
a decrease in regulatory CD4 T cells;
a significant remodeling of gene expression in tumor cells, characterized by:
an increase in the expression of T cell activation genes, cytotoxic cell genes, pathogen defense genes and NK cell function genes;
an increase in the expression of one or more genes selected from the group of CXCL10, CXCL11, IRF1, GZMK, GZMA, CD3D, PRF1, TBX21, CXCR3, STAT1, CD69, CCL2, GZMB, CD3G, ICOS, CD8A, STAT4, GZMM, CCR2, CD3E and IL15;
an increase in the expression of one or more genes selected from the group of CXCL13, GNLY, GZMH, IFNG, CXCL9, CCLS and ITGAE, or a decrease in the expression of one or more genes selected from the group of VEGFA, IHH, IL17A, PROM1, REN, PF4, TSLP and LAG3.

16. The method of claim 2, wherein said poxvirus is a modified Vaccinia Virus Ankara (MVA).

17. The method of claim 7, wherein said HPV-positive cancer is selected from HPV-positive vulvar and vaginal cancers.

18. The method of claim 8, wherein said cancer is HPV-16 positive squamous cell carcinoma of the head and neck (HPV-16+ SCCHN) or HPV-16 positive vulvar or vaginal cancer.

19. The method of claim 10, wherein said HPV-positive metastatic cancer is HPV-positive metastatic cancer with lymph node metastasis.

20. The method of claim 15, wherein said combination induces within the tumor an increase in the CD8/CD3 ratio or a decrease in the Treg/CD8 ratio.
